(19) United States
(12) Patent Application Publication
(10) Pub. No.: US 2004/0223953 A1
(43) Pub. Date: Nov. 11, 2004

(54) Combined adeno-associated virus and adenovirus cocktail gene delivery system for high efficiency gene expression without eliciting immune response in immuno-competent subjects

(76) Inventors: Hsiang-Fu Kung, Hong Kong (CN); Yan Chen, Qingdao (CN); K.D.K. Luk, Hong Kong (CN); Marie C.M. Lin, Hong Kong (CN)

Correspondence Address: DICKSTEIN SHAPIRO MORIN & OSHINSKY LLP, 1177 AVENUE OF THE AMERICAS (6TH AVENUE), 41 ST FL., NEW YORK, NY, 10036-2714, US

(21) Appl. No.: 10/801,648

(22) Filed: Mar. 17, 2004

Related U.S. Application Data
(60) Provisional application No. 60/455,188, filed on Mar. 17, 2003.

(51) Int. Cl. A61K 48/00; C12N 15/861

(52) U.S. Cl. 424/93.2; 435/456; 435/325

(57) ABSTRACT

The present invention provides an efficient gene delivery system using Adeno-Associated Viral (AAV) vector in gene therapy. Furthermore, the invention provides a combined AAV and Adenovirus (Adv) cocktail gene delivery system which is even more efficient in in vivo gene delivery and expression without eliciting any significant immune responses in an immunocompetent subject. In particular, the invention provides a therapeutic agent and methods for preventing, treating, managing, or ameliorating various diseases and disorders including, but not limited to, bone diseases, by delivering Bone Morphogenetic Protein 2 (BMP-2) for new bone formation via gene therapy using said system. The invention provides a nucleic acid molecule comprising an AVV vector and a promoter operably linked to a sequence encoding BMP-2; and a nucleic acid molecule comprising an Adv vector and a promoter operably linked to a sequence encoding BMP-2, as well as vectors and host cells comprising said nucleic acid molecules, respectively.

This application claims priority benefit to U.S. provisional application No. 60/455,188 filed Mar. 17, 2003, which is incorporated herein by reference in its entirety.

### 1. FIELD OF THE INVENTION

The present invention relates to a combined Adeno-Associated Virus (AAV) and Adenovirus (Adv) cocktail gene delivery system for high efficiency gene expression. The present invention further relates to a therapeutic agent and methods for high efficiency gene expression system for use in gene therapy without eliciting an immune response in a subject. The present invention provides a composition comprising a first nucleic acid molecule comprising an adeno-associated viral vector comprising a promoter operably linked to a sequence encoding a polypeptide and a second nucleic acid molecule comprising an adenovirus viral vector comprising a promoter operably linked to a sequence encoding the polypeptide. In particular, the present invention relates to a therapeutic agent and methods for preventing, treating, managing, or ameliorating diseases and disorders of all types. Specifically, the present invention relates to a therapeutic agent and methods for preventing, treating, managing, or ameliorating diseases and disorders including but not limited to, bone diseases, by delivering Bone Morphogenetic Protein 2 (BMP-2) for new bone formation via gene therapy using said composition. The present invention provides a nucleic acid molecule comprising an adeno-associated viral vector comprising a promoter operably linked to a sequence encoding BMP-2 protein. The present invention further provides a nucleic acid molecule comprising an adenovirus viral vector comprising a promoter operably linked to a sequence encoding BMP-2 protein. In particular, the present invention provides a composition comprising a first nucleic acid molecule comprising an adeno-associated viral vector comprising a promoter operably linked to a sequence encoding BMP-2 protein and a second nucleic acid molecule comprising an adenovirus viral vector comprising a promoter operably linked to a sequence encoding BMP-2 protein. Moreover, the methods of the present invention comprise administering the composition of the present invention alone, or in combination with standard and experimental treatment methods for preventing, treating, managing, or ameliorating diseases and disorders of all types including but not limited to, bone diseases.

### 2. BACKGROUND OF INVENTION

Recombinant adeno-associated viruses (AAV) vector system is derived from defective parvoviruses, which depend on essential helper functions provided by other viruses, such as adenovirus (Adv), for efficient viral replication and propagation. AAV are relatively small viruses with a DNA genome measuring less than 5 kb. The viral genome consists of two genes that are flanked by two inverted terminal repeats. In recombinant AAV vectors, viral genes are replaced by foreign DNA and flanked by the inverted terminal repeat regions. Wild-type AAV exhibit a unique tropism for a specific region of human chromosome 19. Therefore, unlike the episomal expression of adenoviruses, AAV are integrated into the host genome and have long-term expression. AAV is an appealing vector system for gene therapy. Unlike Adv, it has no etiological association with any known diseases and has been successfully used to establish efficient and long-term gene expression in vivo in a variety of tissues without significant cellular immune responses or toxicity.

Adeno-associated virus (AAV) is a valuable vector for gene therapy (Monahan et al., 2000, Mol. Med. Today 6:433-440). AAV can mediate long-term expression, because it can either integrate into the genome (Flotte and Carter, 1995, Gene Ther. 2:357-362), or stably maintained as a high-molecular-weight species (Xiao et al., 1996). AAV is also able to transduce a broad range of host tissues including both dividing cells and non-dividing cells (Rabinowitz et al., 1998, Curr. Opin. Biotechnol., 9:470-475). Furthermore, recombinant AAV vectors are a safe mode of gene transfer as infection with wild-type AAV has not been associated with any human disease (Muzyczka, 1992, Curr. Top. Microbiol. Immunol., 158:97-129). More importantly, recombinant AAV contains no viral gene (Samulski et al., 1989, Virol. 63:3822-3828) and expression of genes delivered by these vectors does not activate cell-mediated immunity (Xiao et al., 1996, J. Virol. 70:8098-8108); therefore, AAV vectors can be directly applied to immuno-competent animals or humans.

There are several disadvantages associated with AAV vectors. The cost of producing high titer AAV that is free of Adv contaminations is relatively high, while the efficacy for gene delivery and expression is relatively low. This is in contrast to the adenoviral vectors which are highly efficient and capable of infecting a significant proportion of cells. However, adenovirus is associated with immune rejection, cellular toxicity and inflammatory reactions, which has limited its uses.

Bone regeneration for fracture repair and segmental bone defect healing may be the first major attempted procedure in orthopedic surgery. Autogenous bone graft is currently the major treatment for bone repair. However, it is greatly limited in volume and its harvesting can involve substantial donor site morbidity. Allograft bone has potential for antigenicity and disease transmission, and alloplastic materials have increased infection and extrusion rates, and poor biomechanical properties. Currently the standard approaches to bone regeneration, including internal fixation, autogenous or allogeneic bone graft, and alloplastic material graft, have many disadvantages. Although a variety of pre-clinical studies reported that bone morphogenetic proteins (BMPs) are promising for promoting fracture repair and bone healing (Wozney and Rosen, 1998, Clin. Orthop. 346:26-37), the inability to identify a suitable delivery system, the requirement for large doses, the short half-life and, thereby short-term bio-availability, greatly limited their application to clinical trials. Recombinant BMP2, BMP4, BMP5, BMP7 and BMP9 proteins all have the potential to initiate the osteoinductive cascade (Sakou, 1998, Bone 22:591-603). Unfortunately, the requirement of large doses, short half-life and thus short-term bioavailability of BMPs, lack of practical method for sustained delivery of these exogenous proteins greatly limited the application of BMPs in clinical settings. Gene therapy provides an alternative method for the delivery of BMP protein into tissues for short-term or long-term therapy, a capacity of which may be used to maximally stimulate osteogenesis (Chen, 2001, J. Orthop. Sci. 6:199-207). Gene therapy also allows the targeted delivery of protein to specific cells, and increases the efficacy of the desired protein at specific target site (Sandhu et al., 1999, Bone 24:217-227). Currently available vectors for BMP gene therapy include plasmid (Fang et al., 1996, Proc. Natl. Acad. Sci. 93:5753-5758; Bonadio et al., 1999, Nat. Med. 5:753-759), adenovirus vectors (Alden et al., 1999, Hum. Gene. Ther. 10:2245-2253; Lieberman et al., 1999, J. Bone. Joint. Surg. 81A:905-917; Chen et al., 2002, Biochem. Biophys. Res. Commun. 98:121-127) and retrovirus vectors (Breitbart et al., 1999, Ann. Plast. Surg. 42:488-495; Peng et al., 2001, Mol. Ther. 4:95-104). However, low transfection efficiency of plasmid vectors, immunogenicity and toxicity of adenovirus vectors, and risk for randomly inserted mutation of retrovirus vectors have greatly limited their further applications into humans.

Several delivery system include the used of naked DNA, retroviral vector producers, or adenoviral vectors. Unfortunately, the transfer of naked DNA is typically an inefficient process, especially for systemic use and retroviral vectors may be impractical for human use, while adenoviral-mediated gene transfer is complicated by a host immune response to transduced target cells and its dose-dependent toxicity. Gene therapy however, may represent a better and more promising strategy than direct administration of a BMP protein to maximally stimulate osteogenesis in animals as well as in humans (Chen, 2001, J. Orthop. Sci. 6:199-207).

Previous reports have suggested that BMP gene therapy could be applied for in vivo formation of new bones. However, these studies were conducted either using immuno-deficient animals to avoid immunogenicity against adenovirus vectors, or using ex vivo gene transfer technique which is much more difficult to handle.

Due to the limitations associated with AAV and Adv delivery vehicles, there is a need for novel approaches to delivering gene products to the desired anatomic region. In other words, there is a need for an ideal vector for gene therapy which provides greater efficacy and reduced toxicity over currently available agents.

### 3. SUMMARY OF THE INVENTION

The present invention is based, in part, on the observations by the present inventors that an efficient AAV-mediated BMP2 gene delivery provides a therapeutic benefit in achieving in vivo new bone formation in normal immuno-competent Sprague-Dawley rats (SD rats). Furthermore, more efficient in vivo gene delivery could be achieved using combinational gene transfer using AAV-BMP2 and Ad-BMP2 vectors. In particular, the present inventors discovered that AAV-BMP2-mediated gene delivery efficiency could be further enhanced by introducing low-level Ad-BMP2 without inducing severe immune responses in a host. This combined AAV-BMP2 and Ad-BMP2 gene therapy is useful in inducing bone formation. Thus, the present invention is useful for the treatment of fracture nonunion, segmental bone defects, spinal fusion, or other diseases or disorders that require bone augmentation.

AAV is a nonpathogenic, helper-dependent member of the parvovirus family with several major advantages as a gene-delivery vehicle, such as stable integration, low immunogenicity, long-term expression of the delivered gene, and the ability to infect both dividing and non-dividing cells. It is capable of directing long-term transgene expression in largely terminally differentiated tissues in vivo without causing toxicity to the host and without eliciting a cellular immune response against the transduced cells (Ponnazhagan S et al., 2001, Cancer Res. 61:6313-6321; Lai C C et al., 2001, Invest. Ophthalmol. Vis. Sci. 42(10):2401-7; Nguyen J T et al., 1998, Cancer Research 58:5673-7). Adeno-associated virus (AAV) is a replication-defective virus without any association with immunogenicity and human disease.

An AAV vector carrying human BMP2 gene was constructed. A relatively high dosage of AAV-BMP2 vector (i.e., 10¹² viral particles, VP) was directly injected into the hind limb muscle of immuno-competent Sprague-Dawley rats. Significant new bone formation was visible under X-ray films as early as three weeks post-injection. The ossification tissue was further confirmed by histological staining. Accordingly, the present invention provides AAV-based BMP2 gene therapy for new bone formation in immunocompetent animals. Specifically, the present invention provides orthotopic new bone formation induced by in vivo gene therapy using AAV based bone morphogenetic protein-2 (BMP2) vectors. Mouse myoblast cells (C2C12) transduced with this vector could produce and secrete biologically active BMP2 protein and induced osteogenic activity.

The present invention also provides another efficient in vivo gene-delivery system using combinational gene transfer of AAV-BMP2 and Ad-BMP2 vectors. AAV-BMP2 and low-level Ad-BMP2 were co-injected into the muscle of immuno-competent rats for bone formation. Radiographic examination demonstrated that animals injected with AAV-BMP2 alone had induced only mild ossification, whereas those co-injected with Ad-BMP2 showed a much more significant bone induction. Histological analysis revealed an enlarged medullary cavity without pronounced infiltration of lymphocytes in co-injected rats. Accordingly, the present invention provides AAV-BMP2-mediated gene delivery enhanced by introducing low-level Ad-BMP2 without severe immunogenicity. This combined AAVBMP2 and Ad-BMP2 gene therapy is useful in inducing bone formation.

Accordingly, the present invention provides a therapeutic agent for preventing, treating, managing, or ameliorating diseases or disorders, including, but not limited to, bone diseases. Specifically, the invention provides a therapeutic agent for treating bone diseases, in particular, for bone regeneration to ameliorate fracture repair and segmental bone defect healing, by way of gene therapy. The therapeutic agent of the present invention comprises a nucleic acid molecule containing a nucleotide sequence which encodes a bone morphogenetic protein, or a biologically functional fragment, analog, or variant thereof, in an appropriate vector. In particular, the present invention provides a nucleic acid molecule which comprises a nucleotide sequence encoding an amino acid sequence of SEQ ID NO:2 or a biologically functional fragment, analog, or variant thereof, in an appropriate vector. In a specific embodiment, said vector comprises an expression cassette which comprises an adeno-associated viral vector (AVV) containing chicken beta-actin promoter with cytomegalovirus (CMV) enhancer (CAG promoter) which is operatively linked to the nucleotide sequence encoding the bone morphogenetic protein having an amino acid sequence of SEQ ID NO:2 or a biologically functional fragment, analog, or variant thereof. In a specific embodiment, the vector further comprises the bovine growth hormone polyadenylation signal. In another specific embodiment, the expression cassette was flanked by 145 base pair inverted terminal repeats (ITRs). In one embodiment, said nucleotide sequence has a nucleotide sequence of SEQ ID NO:1. In another embodiment, said nucleotide sequence has a nucleotide sequence that hybridizes under stringent conditions, as herein defined, to a complement of the nucleotide sequence of SEQ ID NO.1, wherein said nucleotide sequence encodes proteins or polypeptides which exhibit at least one structural and/or functional feature of bone morphogenetic protein. In yet another embodiment, said nucleotide sequence has a first nucleotide sequence that hybridizes under stringent conditions to a complement of a second nucleotide sequence encoding an amino acid sequence of SEQ ID NO:2 or a fragment thereof, wherein the first nucleotide sequence encodes proteins or polypeptides which exhibit at least one structural and/or functional feature of bone morphogenetic protein.

The present invention also provides a pharmaceutical composition comprising the therapeutic agent of the present invention and a pharmaceutically acceptable carrier. Such compositions can further include additional active agents. The methods of the present invention further comprise one or more other treatment methods such as orthopedic surgery, fracture repair, segmental bone defects healing, and bone graft. In other specific embodiments, the method of the present invention for the treatment of tumor and/or cancer further comprises surgery, standard and experimental chemotherapies, hormonal therapies, biological therapies/immunotherapies and/or radiation therapies.

Furthermore, the present invention provides a method of preventing, treating, managing, or ameliorating various tumors and/or cancers in a subject, comprising administering to the subject a prophylactically or therapeutically effective amount of the therapeutic agent of the present invention. The tumors and/or cancers may be either primary or metastasized. In one aspect, the therapeutic agent of the present invention is administered to the subject systemically, for example, by intravenous, intramuscular, or subcutaneous injection, or oral administration. In another aspect, the therapeutic agent is administered to the subject locally, for example, by injection to a local blood vessel which supplies blood to a particular organ, tissue, or cell afflicted by disorders or diseases, or by spraying or applying suppository onto afflicted areas of the body.

3.1. Definitions

The term “analog,” especially “bone morphogenetic protein analog” as used herein refers to any member of a series of peptides or nucleic acid molecules having a common biological activity, including antigenicity/immunogenicity and antiagiogenic activity, and/or structural domain and having sufficient amino acid or nucleotide sequence identity as defined herein. Bone morphogenetic protein analog can be from either the same or different species of animals.

As used herein, the term “bone morphogenetic protein” refers to a bone morphogenetic protein from any species. Bone morphogenetic protein may be from primates, including human, or non-primates, including porcine, bovine, mouse, rat, and chicken, etc. One example of bone morphogenetic protein comprises the amino acid sequence of SEQ ID NO:2. Bone morphogenetic protein also refers to a functionally active bone morphogenetic protein (i.e., having bone morphogenetic activity as assessed by the methods as described in Sections 7.2, 7.3, and 7.4) fragments, derivatives and analogs thereof. Bone morphogenetic proteins useful for the present invention includes bone morphogenetic proteins comprising or consisting of the amino acid sequence of SEQ ID NO:2 or having an amino acid sequence comprising substitutions, deletions, inversions, or insertions of one, two, three, or more amino acid residues, consecutive or non-consecutive, with respect to SEQ ID NO:2 and retaining bone morphogenetic activity; and naturally occurring variants of mouse bone morphogenetic protein. Particularly useful bone morphogenetic protein is human bone morphogenetic protein.

The term “variant” as used herein refers either to a naturally occurring allelic variation of a given peptide or a recombinantly prepared variation of a given peptide or protein in which one or more amino acid residues have been modified by amino acid substitution, addition, or deletion.

The term “derivative” as used herein refers to a variation of given peptides or proteins that are otherwise modified, i.e., by covalent attachment of any type of molecule, preferably having bioactivity, to the peptide or protein, including non-naturally occurring amino acids.

The “fragments” described herein include a peptide or polypeptide comprising an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous 10 amino residues, at least 70 contiguous amino acid residues, at least contiguous 80 amino acid residues, at least contiguous 90 amino acid residues, at least contiguous 100 amino acid residues, at least contiguous 125 amino acid residues, at least 150 contiguous amino acid residues, at least contiguous 175 amino acid residues, at least contiguous 200 amino acid residues, at least contiguous 250 amino acid residues, or at least 300 amino acid residues of the amino acid sequence of a polypeptide, preferably that has bone morphogenetic activity.

An “isolated” nucleic acid molecule is one which is separated from other nucleic acid molecules which are present in the natural source of the nucleic acid molecule. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. In a preferred embodiment of the invention, nucleic acid molecules encoding polypeptides/proteins of the invention are isolated or purified. The term “isolated” nucleic acid molecule does not include a nucleic acid that is a member of a library that has not been purified away from other library clones containing other nucleic acid molecules.

As used herein, “in combination” refers to the use of more than one prophylactic and/or therapeutic agents.

As used herein, the terms “manage,” “managing” and “management” refer to the beneficial effects that a subject derives from a prophylactic or therapeutic agent, which do not result in a cure of the disease or disorder. In certain embodiments, a subject is administered one or more prophylactic or therapeutic agents to “manage” a disease or disorder so as to prevent the progression or worsening of the disease or disorder.

As used herein, the terms “prevent,” “preventing” and “prevention” refer to the prevention of a disease or disorder in a subject resulting from the administration of a prophylactic or therapeutic agent.

As used herein, a “prophylactically effective amount” refers to that amount of the prophylactic agent sufficient to prevent a disease or disorder associated with a cell population and, preferably, results in the prevention in proliferation of the cells. A prophylactically effective amount may refer to the amount of prophylactic agent sufficient to prevent the proliferation of cells in a patient.

As used herein, the phrase “side effects” encompasses unwanted and adverse effects of a prophylactic or therapeutic agent. Adverse effects are always unwanted, but unwanted effects are not necessarily adverse. An adverse effect from a prophylactic or therapeutic agent might be harmful or uncomfortable or risky.

The term “under stringent condition” refers to hybridization and washing conditions under which nucleotide sequences having at least 70%, at least 75%, at least 80%, at least 85%, at least 90%, or at least 95% identity to each other remain hybridized to each other. Such hybridization conditions are described in, for example but not limited to, Current Protocols in Molecular Biology, John Wiley & Sons, N.Y. (1989), 6.3.1-6.3.6.; Basic Methods in Molecular Biology, Elsevier Science Publishing Co., Inc., N.Y. (1986), pp. 75-78, and 84-87; and Molecular Cloning, Cold Spring Harbor Laboratory, N.Y. (1982), pp. 387-389, and are well known to those skilled in the art. A preferred, non-limiting example of stringent hybridization conditions is hybridization in 6× sodium chloride/sodium citrate (SSC), 0.5% SDS at about 68° C. followed by one or more washes in 2×SSC, 0.5% SDS at room temperature. Another preferred, non-limiting example of stringent hybridization conditions is hybridization in 6×SSC at about 45° C. followed by one or more washes in 0.2×SSC, 0.1% SDS at about 50-65° C. Yet another preferred, non-limiting example of stringent hybridization conditions is to employ during hybridization a denaturing agent such as formamide, for example, 50% (vol/vol) formamide with 0.1% bovine serum albumin/0.1% Ficoll/0.1% polyvinylpyrrolidone/50 mM sodium phosphate buffer at pH 6.5 with 750 mM NaCl, 75 mM sodium citrate at 42° C.; or to employ 50% formamide, 5×SSC (0.75 M NaCl, 0.075 M Sodium pyrophosphate, 5× Denhardt's solution, sonicated salmon sperm DNA (50 μg/ml), 0.1% SDS, and 10% dextran sulfate at 42° C., with washes at 42° C. in 0.2×SSC and 0.1% SDS.

As used herein, the terms “subject” and “patient” are used interchangeably. As used herein, a subject is preferably a mammal such as a non-primate (e.g., cows, pigs, horses, cats, dogs, rats etc.) and a primate (e.g., monkey and human), most preferably a human.

As used herein, the terms “therapeutic agent” and “therapeutic agents” refer to any agent(s) that can be used in the prevention, treatment, or management of diseases or disorders associated with a cell population. The term “therapeutic agent” refers to a composition comprising one or more vectors of the present invention encoding bone morphogenetic protein.

As used herein, a “therapeutically effective amount” refers to that amount of the therapeutic agent sufficient to treat, manage, or ameliorate a disease or disorder associated with a cell population. A therapeutically effective amount may refer to the amount of therapeutic agent sufficient to increase the number of cells (e.g., promote cell growth). A therapeutically effective amount may also refer to the amount of the therapeutic agent that provides a therapeutic benefit in the treatment or management of a disease or disorder associated with a cell population. Further, a therapeutically effective amount with respect to a therapeutic agent of the invention means that amount of therapeutic agent alone, or in combination with other therapies, that provides a therapeutic benefit in the treatment, management, or amelioration of a disease or disorder associated with a targeted cell population.

As used herein, the terms “therapies” and “therapy” can refer to any protocol(s), method(s) and or agent(s) that can be used in the prevention, treatment, or management of diseases or disorders associated with a cell population.

As used herein, the terms “treat,” “treating” and “treatment” refer to promoting growth of cells that are related to a disease or disorder resulting from the administration of one or more prophylactic or therapeutic agents.

### 4. BRIEF DESCRIPTION OF THE FIGURES

The following figures illustrate the embodiments of the invention and are not meant to limit the scope of the invention encompassed by the claims. 30

FIGS. 1A and 1B show the nucleotide sequence (SEQ ID NO:1) and amino acid sequence (SEQ ID NO:2), respectively, of human BMP2.

FIG. 2 shows a schematic diagram of recombinant AAV-BMP2 vector: ITR, inverted terminal repeat; CAG, chicken β-actin promoter and cytomegalovirus enhancer; MCS, multicloning site; BMP2, human BMP2 cDNA; WPRE, woodchuck hepatitis B virus post-regulatory element; BGH poly A, bovine growth hormone polyadenylation signal.

FIGS. 3A and 3B show an immunofluorescence analysis for BMP2 expression. Immuno-fluorescence of BMP2 protein was analyzed in C2C12 cells infected with AAV-BMP2 at a MOI (Multiplicity Of Infection) of 10⁶ (viral particles/cell) for 24 h. The expressed BMP2 protein is mainly located in the cytoplasm after immunostaining with hBMP2 antibody (FIG. 3A). Those uninfected C2C12 cells showed negative staining (FIG. 3B). Original magnification ×40.

FIG. 4 shows quantification of BMP2 protein in the conditioned medium by ELISA. On day 6 after infection of C2C12 cells with AAV-BMP2 or AAV-EGFP at an MOI (viral particles/cell) of 10⁶, the amount of BMP2 protein in the conditioned medium was measured by ELISA. Values represent mean±S.D (n=3) and were statistically significantly different from the control (asterisk denotes p<0.001) as determined by two-tailed Student's test.

FIGS. 5A and 5B show phenotype changes in C2C12 cells transduced with AAVBMP2. Phenotype changes were observed under conventional microscopy on day 6 after infection with AAV-BMP2 or AAV-EGFP at a MOI of 10⁶ (viral particles/cell). C2C12 cells infected with AAV-BMP2 showed unfused mononuclear round or polygonal phenotype (FIG. 5A). C2C12 cells infected with AAV-EGFP showed significant multinucleate myotube formation (FIG. 5B).

FIG. 6 shows quantification of alkaline phosphatase activity (ALP) in C2C12 cell lysates. Six days after infection of C2C12 cells with AAV-BMP2 or AAV-EGFP at different MOI (particles/cell), the ALP activity was measured from cell layers using pNPP hydrolosis method. Values represent mean±S.D. (n=3) and were statistically significantly different from those of the uninfected cells (asterisks denote p<0.001) as determined by two-tailed Student's test.

FIGS. 7A and 7F show radiographic analysis of new bone formation in immunocompetent SD rats. Significant bone formation could be detected by X-ray film in animals injected with AAV-BMP2. No bone was formed in those rats receiving AAV-EGFP or empty AAV. FIG. 7A: 3 weeks post-injection of high-titer AAV-BMP2 (10¹² VP); FIG. 7B: 8 weeks post-injection of high-titer AAV-BMP2 (10¹² VP); FIG. 7C: 3 weeks post-injection of high-titer AAV-BMP2 (10¹² VP); FIG. 7D: 8 weeks post-injection of high-titer AAV-BMP2 (10¹² VP); FIG. 7E: 8 weeks post-injection of low-titer AAV-BMP2 (5×10¹¹ VP); FIG. 7F: 8 weeks post-injection of AAV-EGFP (10¹² VP). Arrows indicate the regional newly formed bone tissue.

FIGS. 8A-8E show Histological analysis of in vivo bone formation. At 1 week post-injection, there was a significant accumulation of chondrocytes (arrow) with an expanding extracellular cartilaginous matrix within skeletal muscle (M), with mesenchymal cells (arrowhead) surrounding the cartilaginous mass (FIG. 8A: ×10 magnification; FIG. 8B: ×20 magnification). At 3 weeks post-injection, a well-defined ossicification tissue within skeletal muscle (M) was formed, with an obvious cortical rim with trabecular structure (T), medullary cavity containing bone marrow and adipocytes-like cells (+), osteocytes (arrow), osteoblasts (solid arrowhead), multinucleated osteoclasts (open arrowhead), and fatty degeneration (asterisk) in adjacent muscle tissue (FIG. 8C: ×10 magnification; FIG. 8D: ×20 magnification). At 8 weeks post-injection, more mature bone was noticed, with highly ordered lamellar structure, enlarged medullary cavity containing more adipocyte-like cells (+), more osteocytes (arrow), and fatty degeneration (asterisk) in adjacent muscle tissue (FIG. 8E: ×10 magnification; FIG. 8F: ×20 magnification).

FIGS. 9A-9F show immunohistochemical analysis of in vivo BMP2 expression. At 1 week post-injection, BMP2 was mainly expressed in the cytoplasm of chondrocytes (open arrow) within cartilaginous matrix (FIG. 9A: ×20 magnification; FIG. 9B: ×40 magnification). At 3 weeks post-injection, positive staining was mainly detected in osteoblasts (open arrow) within woven bone area. Note that no significant staining developed in differentiated osteocytes (solid arrow) in woven bone matrix (FIG. 9C: ×20 magnification; FIG. 9D: ×40 magnification). AT 8 weeks post-injection, BMP2 was only expressed in osteoblasts (open arrow) within highly organized lamellar cortical bone matrix, and no positive staining was detected in highly matured osteocytes (solid arrow) (FIG. 9E: ×20 magnification; FIG. 9F: ×40 magnification).

FIGS. 10A-10C show a radiographic analysis for new bone formation. At eight weeks post-injection, rats were taken X-ray films for evidence of bone induction. Note that the bone mass in animals injected with both AAV-BMP2 and Ad-BMP2 displayed a significant increase in bone forming area and intensity, comparing to that obtained in rats treated with AAV-BMP2 alone. No radiographic ossification was detected in the rats receiving AAV-EGFP. FIG. 10A: 8 weeks post-injection of AAV-BMP2 plus Ad-BMP2; FIG. 10B: 8 weeks post-injection of AAV-BMP2 alone; FIG. 10C: 8 weeks post-injection of AAV-EGFP. Arrows indicate the regional newly formed bone tissue.

FIG. 11 shows morphometric analysis for radiographic bone forming area. At eight weeks post-injection, the relative bone forming area on X-ray films were measured using ImageQuant software. Values represent mean±S.D. and were significantly different between AAV alone and AAV+Ad (p<0.001), as determined by two-tailed Student's test.

FIG. 12 shows morphometric analysis for radiographic bone forming intensity. At eight weeks post-injection, the relative bone forming intensity on X-ray films were measured using ImageQuant software. Values represent mean±S.D. and were significantly different between AAV alone and AAV+Ad (p<0.001), as determined by two-tailed Student's test.

FIGS. 13A and 13B show histological analysis for new bone formation. At eight weeks post-injection, new bone tissues were harvested and subjected to histological analysis. Hematoxylin and eosin staining demonstrated a well-defined cortical rim (arrowhead), trabeculae structure (arrows), and an enlarged medullary cavity containing bone marrow cells and adipocyte-like cells (+). Note that no significant infiltration of lymphocytes was observed (FIG. 13A: ×5 magnification; FIG. 13B: ×10 magnification).

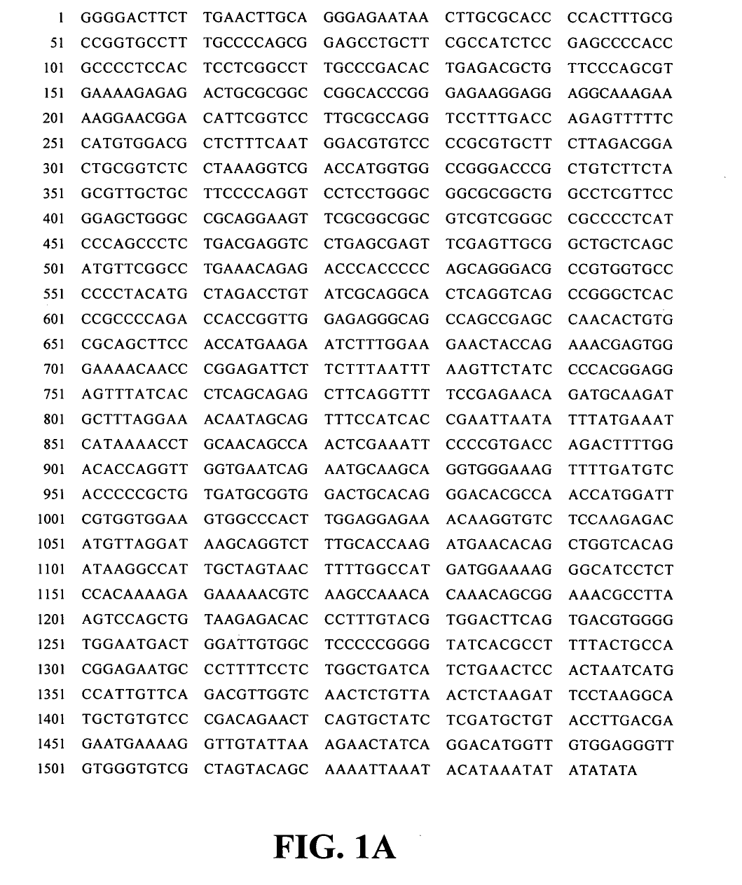

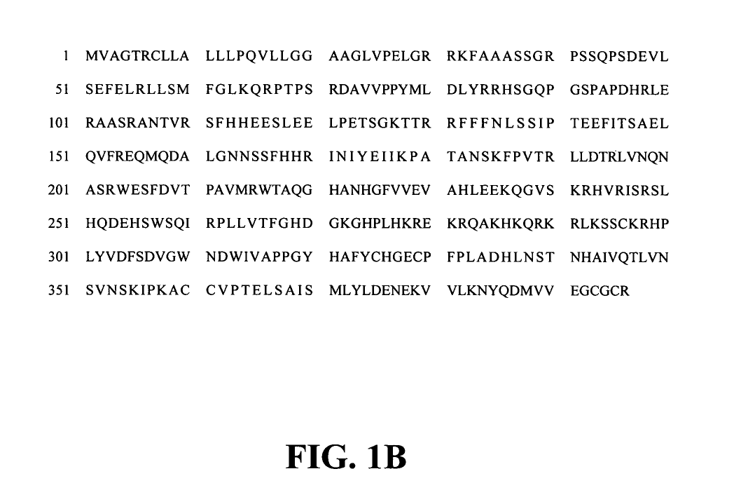

### 5. DETAILED DESCRIPTION OF THE INVENTION

The present invention is directed to using AAV based bone morphogenetic protein-2 (BMP2) vectors to induce orthotopic new bone formation by in vivo gene therapy. An AAV vector carrying human BMP2 gene was constructed and mouse myoblast cells (C2C12) were transduced with this vector. Biologically active BMP2 protein was produced and secreted, which induced osteogenic activity as confirmed by ELISA and alkaline phosphatase activity assay. For in vivo study, AAV-BMP2 vector were directly injected into the hind limb muscle of immuno-competent Sprague-Dawley rats. Significant new bone formation was visible under X-ray films as early as three weeks post-injection. Accordingly, the present invention provides an AAV based BMP2 gene therapy for new bone formation in immuno-competent animals.

The present invention is also directed to a combined Adeno Associated Virus (AAV) plus Adenovirus (Adv) cocktail gene delivery system that achieves a level of gene expression higher than that of AAV alone. This is accomplished without eliciting detectable cytotoxic or undesirable immune responses seen in the adenovirus- and retrovirus-based vectors in vivo in immuno-competent animals. Similar gene delivery by combined AAV and Adv cocktail vectors can be used for other gene therapy cases.

The combined AAV and Adv cocktail gene therapy offers the following advantages: (1) The combined AAV and Adv cocktail gene therapy is more cost effective than AAV gene therapy. High titer of AAV free of Adv is expensive and difficult to produce. Based on the present invention, the cost of therapy can be reduced by at least two mechanisms. Firstly, there is no need to remove the entire Adv virus as long as the Adv level is within the immuno-tolerant level (e.g. less than 5×10⁸ particles of Adv in rat muscle). Therefore, more cost effective wild-type Adv-dependent AAV producer cell lines, which are cost effective, can be used. Secondly, a reduced level of AAV can be used to achieve optimal therapeutic results; (2) The combined AAV and Adv cocktail gene therapy provides higher efficacy as compared to AAV single vector gene therapy. For example, a reduced AAV plus minute dose of Adv combinational gene therapy produces approximately 50% higher efficacy than that of the high titer AAV therapy in the delivery of BMP2 gene product in rat muscle; and (3) No detectable immune responses were observed using the combined-vector system compared to single Adv vector system. Therefore, it can be used as a safe alternative to the more commonly used Adv vector system.

5.1. Construction of Vectors Encoding Bone Morphogenetic Protein-2

The present invention relates to nucleic acid molecules comprising sequences encoding bone morphogenetic proteins. The present invention relates to nucleic acid molecules that encode and direct the expression of bone morphogenetic proteins in appropriate host cells.

Due to the inherent degeneracy of the genetic code, other polynucleotides comprising nucleotide sequences that encode the same amino acid sequence for bone morphogenetic molecule may be used in the practice of the present invention. These include but are not limited to nucleotide sequences comprising all or portions of the coding region of the bone morphogenetic gene which are altered by substitution of different codons that encode the same amino acid residues within the sequence, thus producing a silent change. Such nucleic acid molecule comprises a nucleic acid sequence which hybridizes to sequence or its complementary sequence encoding the bone morphogenetic gene under stringent conditions. The phrase “stringent conditions” as used herein refers to those hybridizing conditions that (1) employ low ionic strength and high temperature for washing, for example, 0.015 M NaCl/0.0015 M sodium citrate/0.1% SDS at 50° C.; or hybridization in 6× sodium chloride/sodium citrate (SSC), 0.5% SDS at about 68° C. followed by one or more washes in 2×SSC, 0.5% SDS at room temperature; or hybridization in 6×SSC at about 45° C. followed by one or more washes in 0.2×SSC, 0.1% SDS at about 50-65° C.; (2) employ during hybridization a denaturing agent such as formamide, for example, 50% (vol/vol) formamide with 0.1% bovine serum albumin/0.1% Ficoll/0.1% polyvinylpyrrolidone/50 mM sodium phosphate buffer at pH 6.5 with 750 mM NaCl, 75 mM sodium citrate at 42° C.; or (3) employ 50% formamide, 5×SSC (0.75 M NaCl, 0.075 M Sodium pyrophosphate, 5× Denhardt's solution, sonicated salmon sperm DNA (50 μg/ml), 0.1% SDS, and 10% dextran sulfate at 42° C., with washes at 42° C. in 0.2×SSC and 0.1% SDS.

The nucleic acid molecules comprising sequences encoding bone morphogenetic molecules may be engineered, including but not limited to, alterations which modify processing and expression of the gene product. For example, to alter glycosylation patterns or phosphorylation, etc.

In order to express a biologically active bone morphogenetic protein, the nucleotide sequence encoding bone morphogenetic protein is inserted into an appropriate expression vector, i.e., a vector which contains the necessary elements for the transcription and translation of the inserted nucleic acid molecule. The gene products as well as host cells or cell lines transfected or transformed with recombinant expression vectors are within the scope of the present invention.

Methods which are well known to those skilled in the art can be used to construct expression vectors containing the sequence that encodes the bone morphogenetic protein and appropriate transcriptional/translational control signals. These methods include in vitro recombinant DNA techniques, synthetic techniques and in vivo recombination/genetic recombination. See, for example, the techniques described in Sambrook et al., 1989, Molecular Cloning A Laboratory Manual, Cold Spring Harbor Laboratory, N.Y.; and Ausubel et al., 1989, Current Protocols in Molecular Biology , Greene Publishing Associates and Wiley Interscience, N.Y.

A variety of host-expression vector systems may be utilized to express the bone morphogenetic protein. These include but are not limited to microorganisms such as bacteria transformed with recombinant bacteriophage DNA, plasmid DNA or cosmid DNA expression vectors; yeast transformed with recombinant yeast expression vectors; insect cell systems infected with recombinant virus expression vectors (e.g., baculovirus); plant cell systems infected with recombinant virus expression vectors (e.g., cauliflower mosaic virus, CaMV; tobacco mosaic virus, TMV) or transformed with recombinant plasmid expression vectors (e.g., Ti plasmid); or animal cell systems. Examples of a preferred host-expression vector system are illustrated below.

The expression elements of each system vary in their strength and specificities. Depending on the host/vector system utilized, any of a number of suitable transcription and translation elements, including constitutive and inducible promoters, may be used in the expression vector. For example, when cloning in bacterial systems, inducible promoters such as pL of bacteriophage λ, plac, ptrp, ptac (ptrp-lac hybrid promoter; cytomegalovirus promoter) and the like may be used; when cloning in insect cell systems, promoters such as the baculovirus polyhedrin promoter may be used; when cloning in plant cell systems, promoters derived from the genome of plant cells (e.g., heat shock promoters; the promoter for the small subunit of RUBISCO; the promoter for the chlorophyll α/β binding protein) or from plant viruses (e.g., the 35S RNA promoter of CaMV; the coat protein promoter of TMV) may be used; when cloning in mammalian cell systems, promoters derived from the genome of mammalian cells (e.g., metallothionein promoter), from mammalian viruses (e.g., the adenovirus late promoter; the vaccinia virus 7.5K promoter), or avian cells (e.g., chicken beta-actin promoter) may be used; when generating cell lines that contain multiple copies of the chimeric DNA, SV40-, BPV- and EBV-based vectors may be used with an appropriate selectable marker.

In bacterial systems a number of expression vectors may be advantageously selected depending upon the use intended for the protein expressed. For example, when large quantities of protein are to be produced, vectors which direct the expression of high levels of protein products that are readily purified may be desirable. Such vectors include but are not limited to the pHL906 vector (Fishman et al., 1994, Biochem. 33:6235-6243), the E. coli expression vector pUR278 (Ruther et al., 1983, EMBO J. 2:1791), in which the protein coding sequence may be ligated into the vector in frame with the lacZ coding region so that a hybrid AS-lacZ protein is produced; pIN vectors (Inouye & Inouye, 1985, Nucleic acids Res. 13:3101-3109; Van Heeke & Schuster, 1989, J. Biol. Chem. 264:5503-5509); and the like.

Specific initiation signals may also be required for efficient translation of the nucleic acid molecule of the present invention. These signals include the ATG initiation codon and adjacent sequences. In cases where the entire gene, including its own initiation codon and adjacent sequences, is inserted into the appropriate expression vector, no additional translational control signals may be needed. However, in cases where the bone morphogenetic protein coding sequence does not include its own initiation codon, exogenous translational control signals, including the ATG initiation codon, must be provided. Furthermore, the initiation codon must be in phase with the reading frame of the bone morphogenetic protein coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., 1987, Methods in Enzymol. 153:516-544).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. The presence of consensus N glycosylation sites in the bone morphogenetic protein may require proper modification for optimal function. Different host cells have characteristic and specific mechanisms for the post-translational processing and modification of proteins. Appropriate cell lines or host systems can be chosen to ensure the correct modification and processing of the protein. To this end, eukaryotic host cells which possess the cellular machinery for proper processing of the primary transcript, glycosylation, and phosphorylation of the bone morphogenetic protein may be used. Such mammalian host cells include but are not limited to CHO, VERO, BHK, HeLa, COS, MDCK, 293, W138, and the like.

For long-term, high-yield production of bone morphogenetic proteins, stable expression is preferred. For example, cell lines which stably express the bone morphogenetic protein may be engineered. Rather than using expression vectors which contain viral origins of replication, host cells can be transformed with a coding sequence controlled by appropriate expression control elements, such as promoter (e.g., chicken beta-actin promoter), enhancer (e.g., CMV enhancer), transcription terminators, posttranscriptional regulatory element (e.g., WPRE), polyadenylation sites, etc., and a selectable marker. Following the introduction of foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines.

A number of selection systems may be used, including but not limited to the herpes simplex virus thymidine kinase (Wigler et al., 1977, Cell 11:223), hypoxanthine-guanine phosphoribosyltransferase (Szybalska & Szybalski, 1962, Proc. Natl. Acad. Sci. USA 48:2026), and adenine phosphoribosyltransferase (Lowy et al., 1980, Cell 22:817) genes can be employed in tk-, hgprt- or aprt-cells, respectively. Also, antimetabolite resistance can be used as the basis of selection for dhfr, which confers resistance to methotrexate (Wigler et al., 1980, Natl. Acad. Sci. USA 77:3567; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Colberre-Garapin et al., 1981, J. Mol. Biol. 150:1); and hygro, which confers resistance to hygromycin (Santerre et al., 1984, Gene 30:147) genes. Additional selectable genes have been described, namely trpB, which allows cells to utilize indole in place of tryptophan; hisD, which allows cells to utilize histinol in place of histidine (Hartman & Mulligan, 1988, Proc. Natl. Acad. Sci. USA 85:8047); and ODC (ornithine decarboxylase) which confers resistance to the ornithine decarboxylase inhibitor, 2-(difluoromethyl)-DL-ornithine, DFMO (McConlogue L., 1987, In: Current Communications in Molecular Biology, Cold Spring Harbor Laboratory ed.).

The identity and functional activities of an bone morphogenetic protein can be readily determined by methods well known in the art. For example, antibodies to the protein may be used to identify the protein in Western blot analysis or immunohistochemical staining of tissues.

5.2. Pharmaceutical Compositions

The therapeutic agent of the invention can be incorporated into pharmaceutical compositions suitable for administration. Such compositions typically comprise the nucleic acid molecule or protein, and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The invention includes methods for preparing pharmaceutical compositions. Such methods comprise formulating a pharmaceutically acceptable carrier with the therapeutic agent of the invention. Such compositions can further include additional active agents. Thus, the invention further includes methods for preparing a pharmaceutical composition by formulating a pharmaceutically acceptable carrier with a polypeptide or nucleic acid of the invention and one or more additional active compounds.

A pharmaceutical composition of the invention is formulated to be compatible with its intended route of administration. Examples of routes of administration include parenteral, e.g., intravenous, intradermal, subcutaneous, transdermal (topical), transmucosal, intra-articular, intraperitoneal, and intrapleural, as well as oral, inhalation, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. A pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL™ (BASF; Parsippany, N.J.) or phosphate buffered saline (PBS). In all cases, the composition must be sterile and should be fluid to the extent that easy injectability with a syringe exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a nucleic acid molecule or polypeptide) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient, such as starch or lactose; a disintegrating agent, such as alginic acid, Primogel, or corn starch; a lubricant, such as magnesium stearate or Sterotes; a glidant, such as colloidal silicon dioxide; a sweetening agent, such as sucrose or saccharin; or a flavoring agent, such as peppermint, methyl salicylate, or orange flavoring.

For administration by inhalation, the compounds are delivered in the form of an aerosol spray from a pressurized container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art. The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography. For the use of animal models to determine optimal dosage, see, for example, Sections 6.3, 6.4, and 6.6 infra.

The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a therapeutic agent, such as nucleic acid molecule, protein, polypeptide, or antibody can include a single treatment or, preferably, can include a series of treatments. It will also be appreciated that the effective dosage of antibody, protein, or polypeptide used for treatment may increase or decrease over the course of a particular treatment. Changes in dosage may result and become apparent from the results of diagnostic assays as described herein.

The nucleic acid molecules of the invention can be inserted into vectors and used as gene therapy vectors. Methods of delivering gene therapy vectors to a subject include: intravenous injection, local administration (U.S. Pat. No. 5,328,470) or by stereotactic injection (see, e.g., Chen, et al., 1994, Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system. With regard to gene therapy, see further discussion in Section 5.3.

5.3. Therapeutic/Prophylactic Methods Using Bone Morphogenetic Protein

The present invention is directed to therapeutic or prophylactic method which leads to the treatment or prevention of a disease or disorder. In one embodiment, the disease or disorder is treatable or preventable by increasing the number of cells or to promote the proliferation of cells. In specific embodiments, the method of the present invention is useful to promote bone formation, wound healing (e.g., burns, incisions, ulcers), tissue repair, nerve cells regeneration, neuronal survival; to stimulate neuronal growth, neural crest cell differentiation, hematopoiesis; to regulate follicle stimulating hormone, lung and kidney cell function; to decrease proliferation of kidney epithelial cells, proliferation of lung epithelial cells; to inhibit kidney tubule formation; to repair cartilage defects; to prevent stroke (by reducing the size of the infarct); and to suppress the development of gonadal tumors.

In specific embodiment, the method of the present invention is used to treat genetic diseases of bone formation (e.g., fibrodysplasia ossificans progressiva (FOP) and progressive osseous heteroplasia (POH)), bone fracture, peridontal diseases, osteoporosis (age-related, post-menopausal hormone status-related, diabetes-related, hyperparathyroidism-related, glucocorticoid-related), osteoblastic metastases, osteoarthritis, arthritis, rheumatism, lower back pain, degenerative disc disease, spinal injury, growth plate injury, neural tumor, glaucoma, tracheomalacia, pulmonary hypertension, kidney diseases (e.g., renal fibrosis), hematopoietic injury, and inflammation (reduces macrophage infiltration).

Other genes that may be produced for the treatment of diseases or disorder of the present invention includes angiogenic factors including acidic and basic fibroblast growth factors, vascular endothelial growth factor, epidermal growth factor, transforming growth factor α and β, platelet-derived endothelial growth factor, platelet-derived growth factor, tumor necrosis factor a, hepatocyte growth factor and insulin like growth factor; growth factors; cell cycle inhibitors, kinase (“TK”) and other agents useful for interfering with cell proliferation, including agents for treating malignancies. Still other useful factors, which can be provided as polypeptides or as DNA encoding these polypeptides, include the family of bone morphogenic proteins (“BMP's”). The known proteins include BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15, and BMP-16. Currently preferred BMP's are any of BMP-2, BMP-3, BMP-4, BMP-5, BMP-6 and BMP-7 (Rengachary et al., 2002, Neurosurg Focus 13 (6):1-6). In addition, molecules capable of inducing an upstream or downstream effect of a BMP can be used. Such molecules include any of the “hedgehog” proteins, or the DNA's encoding them.

Most preferably, gene therapy mediates a therapeutic effect by expressing genes such as thymidine kinase, retinoblastoma, p53, p21, fasL, VEGF, HGF, P16 (INK4a), MTS-1, CDKN2, and others which have demonstrated effectiveness in inhibiting intimal hyperplasia.

### 5.3.1. Therapeutic Administration

The invention provides methods of preventing and treating diseases or disorders by administrating to an animal (e.g., cows, pigs, horses, chickens, cats, dogs, humans, etc.) an effective amount of the polynucleotides of the invention. The polynucleotides of the invention may be administered to a subject per se or in the form of a pharmaceutical composition for the treatment and prevention of diseases or disorders.

In certain embodiments, therapeutic composition of the invention is administered to a mammal, preferably a human, concurrently with one or more other therapeutic composition useful for the treatment of diseases or disorders. The term “concurrently” is not limited to the administration of therapeutic composition at exactly the same time, but rather it is meant that the composition of the present invention and the other agent are administered to a mammal in a sequence and within a time interval such that the composition comprising the polynucleotides can act together with the other composition to provide an increased benefit than if they were administered otherwise. For example, each therapeutic composition may be administered at the same time or sequentially in any order at different points in time; however, if not administered at the same time, they should be administered sufficiently close in time so as to provide the desired therapeutic effect. Each therapeutic composition can be administered separately, in any appropriate form and by any suitable route. In other embodiments, the composition of the present invention is administered before, concurrently or after surgery. In various embodiments, the therapeutic compositions are administered less than 1 hour apart, at about 1 hour apart, at about 1 hour to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, no more than 24 hours apart or no more than 48 hours apart. In preferred embodiments, two or more components are administered within the same patient visit.

In a preferred embodiment, a first composition comprises an adeno-associated viral vector comprising a promoter operably linked to a sequence encoding a polypeptide, a second composition comprises an adenoviral vector comprising a promoter operably linked to a sequence encoding a polypeptide. In another preferred embodiment, a first composition comprises an adenoviral vector comprising a promoter operably linked to a sequence encoding a polypeptide, a second composition comprises an adeno-associated viral vector comprising a promoter operably linked to a sequence encoding a polypeptide.

In other embodiments, the therapeutic compositions are administered at about 2 to 4 days apart, at about 4 to 6 days apart, at about 1 week part, at about 1 to 2 weeks apart, or more than 2 weeks apart. In preferred embodiments, the therapeutic compositions are administered in a time frame where both compositions are still active. One skilled in the art would be able to determine such a time frame by determining the half life of the administered compositions.

The dosage amounts and frequencies of administration provided herein are encompassed by the terms therapeutically effective. The dosage and frequency further will typically vary according to factors specific for each patient depending on the specific therapeutic composition administered, the severity and type of disease or disorder, the route of administration, as well as age, body weight, response, and the past medical history of the patient. Suitable regimens can be selected by one skilled in the art by considering such factors and by following, for example, dosages reported in the literature and recommended in the Physician 's Desk Reference (56th ed., 2002). In preferred embodiments, the number of viral particles per treatment are at most 1×10⁵, 2×10⁵, 5×10⁵, 1×10⁶, 2×10⁶, 5×10⁶, 1×10⁷, 2×10⁷, 5×10⁷, 1×10⁸, 2×10⁸, 5×10⁸, 1×10⁹, 2×10⁹, 5×10⁹, 1×10¹⁰, 2×10¹⁰, 5×10¹⁰, 1×10¹¹, 2×10¹¹, 5×10¹¹, 1×10¹², 2×10¹², 5×10¹², 1×10¹³, 2×10¹³, 5×10¹³, 1×10¹⁴, 2×10¹⁴, 5×10¹⁴, 1×10¹⁵, 2×10¹⁵, 5×10¹⁵, 1×10¹⁶, 2×10¹⁶, 5×10¹⁶. In preferred embodiments, when AAV vectors and Ad vectors are used in combination, the number of viral particles of the AAV vectors are higher than that of the Ad vectors.

Various delivery systems are known and can be used to administer the therapeutic composition of the present invention, e.g., encapsulation in liposomes, microparticles, microcapsules, recombinant cells capable of expressing the polypeptide, receptor-mediated endocytosis (see, e.g., Wu and Wu, J. Biol. Chem. 262:4429-4432 (1987)), construction of a nucleic acid as part of a retroviral or other vector, etc. Methods of administering a therapeutic composition of the invention include, but are not limited to, parenteral administration (e.g., intradermal, intramuscular, intraperitoneal, intravenous and subcutaneous), epidural, and mucosal (e.g., intranasal and oral routes). In a specific embodiment, therapeutic composition of the invention are administered intramuscularly, intravenously, or subcutaneously. The therapeutic composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

In a specific embodiment, it may be desirable to administer the therapeutic composition of the invention locally to the area in need of treatment; this may be achieved by, for example, and not by way of limitation, local infusion, by injection, or by means of an implant, said implant being of a porous, non-porous, or gelatinous material, including membranes, such as sialastic membranes, or fibers.

In yet another embodiment, the therapeutic composition can be delivered in a controlled release or sustained release system. In one embodiment, a pump may be used to achieve controlled or sustained release (see Langer, supra; Sefton, 1987, CRC Crit. Ref. Biomed. Eng. 14:20; Buchwald et al., 1980, Surgery 88:507; Saudek et al., 1989, N. Engl. J. Med. 321:574). In another embodiment, polymeric materials can be used to achieve controlled or sustained release of the therapeutic composition of the invention (see e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J., Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105); U.S. Pat. No. 5,679,377; U.S. Pat. No. 5,916,597; U.S. Pat. No. 5,912,015; U.S. Pat. No. 5,989,463; U.S. Pat. No. 5,128,326; PCT Publication No. WO 99/15154; 20 and PCT Publication No. WO 99/20253. Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In a preferred embodiment, the polymer used in a sustained release formulation is inert, free of leachable impurities, stable on storage, sterile, and biodegradable. In yet another embodiment, a controlled or sustained release system can be placed in proximity of the prophylactic or therapeutic target, thus requiring only a fraction of the systemic dose (see, e.g., Goodson, in Medical Applications of Controlled Release, supra, vol. 2, pp. 115-138 (1984)).

### 5.3.2. Gene Therapy

The present invention provides methods for the treatment of diseases or disorders comprising administering nucleic acid molecules of the present invention encoding bone morphogenetic proteins. In a specific embodiment, nucleic acids comprising sequences encoding bone morphogenetic proteins are administered to treat diseases or disorders, by way of gene therapy. Gene therapy refers to therapy performed by the administration to a subject of an expressed or expressible nucleic acid. In this embodiment of the invention, the nucleic acids produce their encoded protein that mediates a therapeutic effect.

Any of the methods for gene therapy available in the art can be used according to the present invention. Exemplary methods are described below.

For general reviews of the methods of gene therapy, see Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May, 1993, TIBTECH 11(5):155-215). Methods commonly known in the art of recombinant DNA technology which can be used are described in Ausubel et al. (eds.), 1993, Current Protocols in Molecular Biology, John Wiley & Sons, NY; and Kriegler, 1990, Gene Transfer and Expression, A Laboratory Manual, Stockton Press, NY.

In one aspect, a composition comprising nucleic acid sequences encoding bone morphogenetic proteins in expression vectors of the present invention are administered to suitable hosts. The expression of nucleic acid sequences encoding bone morphogenetic proteins may be regulated by any inducible, constitutive, or tissue-specific promoter known to those of skill in the art. In a specific embodiment, the nucleic acid to be introduced for purposes of gene therapy comprises an inducible promoter operably linked to the coding region, such that expression of the nucleic acid is controllable by controlling the presence or absence of the appropriate inducer of transcription.

In a particular embodiment, nucleic acid molecules encoding bone morphogenetic proteins are flanked by regions that promote homologous recombination at a desired site in the genome, thus providing for intrachromosomal expression of said coding regions (Koller and Smithies, 1989, Proc. Natl. Acad. Sci. USA 86:8932-8935; Zijlstra et al., 1989, Nature 342:435-438).

In a specific embodiment, pharmaceutical composition comprising an adeno-associated viral vector comprising a promoter operably linked to a sequence encoding BMP-2 protein is administered to a subject. In another specific embodiment, the pharmaceutical composition further comprises an adenoviral vector comprising a promoter operably linked to a sequence encoding BMP2 protein.

Delivery of the nucleic acids into a patient may be either direct, in which case the patient is directly exposed to the nucleic acid or nucleic acid-carrying vectors, or indirect, in which case, cells are first transformed with the nucleic acids in vitro, then transplanted into the patient. These two approaches are known, respectively, as in vivo or ex vivo gene therapy.

In a specific embodiment, the nucleic acid sequences are directly administered in vivo, where it is expressed to produce the encoded product. This can be accomplished by any of numerous methods known in the art, e.g., by constructing them as part of an appropriate nucleic acid expression vector and administering it so that they become intracellular, e.g., by infection using defective or attenuated retrovirals or other viral vectors (see U.S. Pat. No. 4,980,286), or by direct injection of naked DNA, or by use of microparticle bombardment (e.g., a gene gun; Biolistic, Dupont), or coating with lipids or cell-surface receptors or transfecting agents, encapsulation in liposomes, microparticles, or microcapsules, or by administering them in linkage to a peptide which is known to enter the nucleus, by administering it in linkage to a ligand subject to receptor-mediated endocytosis (see, e.g., Wu and Wu, 1987, J. Biol. Chem. 262:4429-4432) (which can be used to target cell types specifically expressing the receptors), etc. In another embodiment, nucleic acid-ligand complexes can be formed in which the ligand comprises a fusogenic viral peptide to disrupt endosomes, allowing the nucleic acid to avoid lysosomal degradation.

In yet another embodiment, the nucleic acid can be targeted in vivo for cell specific uptake and expression, by targeting a specific receptor (see, e.g., PCT Publications WO 92/06180 dated Apr. 16, 1992 (Wu et al.); WO 92/22635 dated Dec. 23, 1992 (Wilson et al.); WO92/20316 dated Nov. 26, 1992 (Findeis et al.); WO93/14188 dated Jul. 22, 1993 (Clarke et al.), WO 93/20221 dated Oct. 14, 1993 (Young)). Alternatively, the nucleic acid can be introduced intracellularly and incorporated within host cell DNA for expression, by homologous recombination (Koller and Smithies, 1989, Proc. Natl. Acad. Sci. USA 86:8932-8935; Zijlstra et al., 1989, Nature 342:435-438).

In a specific embodiment, viral vectors are used to express nucleic acid sequences. For example, a retroviral vector can be used (see Miller et al., 1993, Meth. Enzymol. 217:581-599). These retroviral vectors have deleted retroviral sequences that are not necessary for packaging of the viral genome and integration into host cell DNA. The nucleic acid sequences encoding the nucleic acid molecules of the invention to be used in gene therapy are cloned into one or more vectors, which facilitates delivery of the gene into a patient. More detail about retroviral vectors can be found in Boesen et al., 1994, Biotherapy 6:291-302, which describes the use of a retroviral vector to deliver the mdr1 gene to hematopoietic stem cells in order to make the stem cells more resistant to chemotherapy. Other references illustrating the use of retroviral vectors in gene therapy are: Clowes et al., 1994, J. Clin. Invest. 93:644-651; Kiem et al., 1994, Blood 83:1467-1473; Salmons and Gunzberg, 1993, Human Gene Therapy 4:129-141; and Grossman and Wilson, 1993, Curr. Opin. in Genetics and Devel. 3:110-114.

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, 1993, Current Opinion in Genetics and Development 3:499-503 present a review of adenovirus-based gene therapy. Bout et al., 1994, Human Gene Therapy 5:3-10 demonstrated the use of adenovirus vectors to transfer genes to the respiratory epithelia of rhesus monkeys. Other instances of the use of adenoviruses in gene therapy can be found in Rosenfeld et al., 1991, Science 252:431-434; Rosenfeld et al., 1992, Cell 68:143-155; Mastrangeli et al., 1993, J. Clin. Invest. 91:225-234; PCT Publication WO94/12649; and Wang, et al., 1995, Gene Therapy 2:775-783. In a preferred embodiment, adenovirus vectors are used. Adeno-associated virus (AAV) has also been proposed for use in gene therapy (Walsh et al., 1993, Proc. Soc. Exp. Biol. Med. 204:289-300; U.S. Pat. No. 5,436,146). In another preferred embodiment, adeno-associated viral vectors are used. In the most preferred embodiment, both adenoviral vectors and adeno-associated viral vectors are used in combination.

Most preferable viral vectors for the present invention are adeno-associated viral (AVV) vectors. AAV vector leads to persistent (>6 months) expression of a transgene in both gut epithelial cells and hepatocytes, resulting in long-term phenotypic recovery in a diabetic animal model (Xu, R A et al., 2001, Perarolly transduction of diffuse cells and hepatocyte insulin leading to euglycemia in diabetic rats, Mol Ther 3:S180; During, M J et al., 1998, Parorally gene therapy of lactose intolerance using an adeno-associated virus vector, Nature Med. 4:1131-1135; During M J et al., 2000, An oral vaccine against NMDAR1 with efficacy in experimental stroke and epilepsy, Science 287:1453-1460).

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a patient.

In one embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells (see, e.g., Loeffler and Behr, 1993, Meth. Enzymol. 217:599-618; Cohen et al., 1993, Meth. Enzymol. 217:618-644; Cline, 1985, Pharmac. Ther. 29:69-92) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a patient by various methods known in the art. Recombinant blood cells (e.g., hematopoietic stem or progenitor cells) are preferably administered intravenously. The amount of cells envisioned for use depends on the desired effect, patient state, etc., and can be determined by one skilled in the art.

Cells into which a nucleic acid can be introduced for purposes of gene therapy encompass any desired, available cell type, and include but are not limited to epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood, fetal liver, etc.

In a preferred embodiment, the cell used for gene therapy is autologous to the patient.

In an embodiment in which recombinant cells are used in gene therapy, nucleic acid sequences of the present invention encoding bone morphogenetic proteins are introduced into the cells such that they are expressible by the cells or their progeny, and the recombinant cells are then administered in vivo for therapeutic effect. In a specific embodiment, stem or progenitor cells are used. Any stem and/or progenitor cells which can be isolated and maintained in vitro can potentially be used in accordance with this embodiment of the present invention (see e.g. PCT Publication WO 94/08598, dated Apr. 28, 1994; Stemple and Anderson, 1992, Cell 71:973-985; Rheinwald, 1980, Meth. Cell Bio. 21A:229; and Pittelkow and Scott, 1986, Mayo Clinic Proc. 61:771).

5.4. Demonstration of Therapeutic or Prophylactic Utility

The compositions of the invention are preferably tested in vitro, and then in vivo for the desired therapeutic or prophylactic activity, prior to use in humans. For example, in vitro assays to demonstrate the therapeutic or prophylactic utility of a composition include, the effect of a composition on a cell line, particularly one characteristic of a cell type in need of the treatment, or a patient tissue sample. The effect of the composition on the cell line and/or tissue sample can be determined utilizing techniques known to those of skill in the art including, but not limited to, those described in Sections 7.2, 7.3, 7.4, and 7.5.

Specifically, myoblast cell line, such as C2C12 may be used to assess the therapeutic effects of the polynucleotides encoding bone morphogenetic protein. Techniques known to those skilled in the art can be used for measuring cell activities. For example, cellular proliferation can be assayed by ³H-thymidine incorporation assays and trypan blue cell counts.

In a specific example, the expression of the therapeutic agent of the present invention can be detected by in situ hybridization using a specific probe, or by Western blotting or immunohistochemical staining using specific antibodies.

In yet another specific example, the therapeutic or prophylactic activity of the present therapeutic agent can be assessed by measuring cell growth, myogenesis, osteogenesis and compare with that of control samples.

In various embodiments, with the invention, in vitro assays which can be used to determine whether administration of a specific composition is indicated, include in vitro cell culture assays in which a patient tissue sample is grown in culture, and exposed to or otherwise administered a composition, and the effect of such composition upon the tissue sample is observed.

Compositions for use in therapy can be tested in suitable animal model systems prior to testing in humans, including but not limited to rats, mice, chicken, cows, monkeys, rabbits, etc. For in vivo testing, prior to administration to humans, any animal model system known in the art may be used.

### 5.4.1. Toxicity

Preferably, a therapeutically effective dose of the polynucleotides described herein will provide therapeutic benefit without causing substantial toxicity.

Toxicity of the proteins encoded by the polynucleotides of the present invention described herein can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. Proteins which exhibit high therapeutic indices are preferred. The data obtained from these cell culture assays and animal studies can be used in formulating a dosage range that is not toxic for use in human. The dosage of the proteins described herein lies preferably within a range of circulating concentrations that include the effective dose with little or no toxicity. The dosage may vary within this range depending upon the dosage form employed and the route of administration utilized. The exact formulation, route of administration and dosage can be chosen by the individual physician in view of the patient's condition. (See, e.g., Fingl et al., 1975, In: The Pharmacological Basis of Therapeutics, Ch. 1, p. 1).

5.5. Kits

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

The present invention also provides kits that can be used in the above methods. In one embodiment, a kit comprises the polynucleotides in one or more containers.

In certain embodiments, the kits of the invention contain instructions for the use of the polynucleotides for the treatment, prevention of diseases or disorders.

### 6. EXAMPLES

The invention having been described, the following examples are offered by way of illustration and not limitation.

6.1. Construction of an Adeno-Associated Virus Carrying the BMP2 Gene

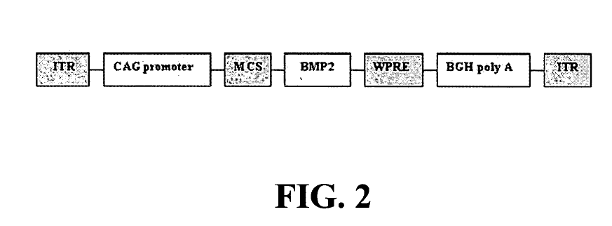

The recombinant AAV2 packaging plasmid, termed pAM/CAG-pL-WPRE-15 BGH-polyA, was constructed by deleting all of the viral open reading frame (ORF), and introducing the chicken β-actin promoter and cytomegalovirus (CMV) enhancer, a multicloning site, and the bovine growth hormone polyadenylation signal. The expression cassette was flanked by 145 base pair inverted terminal repeats (ITRs), which contained palindromic sequences necessary in cis elements for replication of the viral genome. Vector plasmid pAM/CAG-BMP2 was constructed by cloning human BMP2 cDNA into the EcoR I and XhoI sites located between two ITRs (FIG. 2). Vectors pAM/CAG-EGFP was constructed by inserting enhanced green fluorescent protein (EGFP) gene into the EcoR I and XhoI sites. All plasmids DNA for virus packaging were purified by a MaxiPrep Plasmid Preparation Kit (Qiagen, Hilden, Germany).

AAV vectors were produced using a helper virus-free system with some modification (Grimm et al., 1998, Hum Gene Ther 9:2745-2760). Briefly, trypsinized HEK293 cells were plated at 4×10⁶ cells in 150-mm culture dishes containing 20 ml Dulbecco's modified Eagle's medium (DMEM), supplemented with 10% fetal bovine serum (FBS, Gibco BRL, Gaithersburg, Md., USA), 1% penicillin/streptomycin, 1% glutamine, and incubated at 37° C. under 5% CO₂ for 24-48 h. After cells reach 80% confluence, each plate of 293 cells were co-transfected with 11 μg of pAM/CAG-BMP2 (or 11 μg of pAM/CAGEGFP) and 66 μg of AAV helper plasmid pDG by means of calcium phosphate coprecipitation method. After incubation at 37° C. for 10 h, growth medium was replaced with fresh medium and cells were further incubated for approximately 60 h. The transfected 293 cells were trypsinized, harvested by centrifugation, and re-suspended in 150 mM NaCl buffer containing 20 mM Tris at pH 8.0. After two cycles of freezing and thawing followed by centrifugation, supernatants containing AAV vectors were combined and purified by HiTrap Heparin column chromatography (Sigma, St. Louis, Mo., USA). Peak virus fractions were collected and dialyzed against PBS supplemented with 1 mM MgSO₄, and then concentrated by 100K-MicroSep centrifugal concentrator (Life technologies, Carlsbad, Calif., USA). The AAV viral genome titer was quantified by Real-Time PCR using TaqMan (Perkin-Elmer Biosystems, Foster City, Calif., USA). The recombinant AAV-EGFP was also constructed by the same procedure.

6.2. Expression of Cellular BMP2 in Myoblast C2C12 Cells

For immuno-fluorescence analysis of cellular BMP2 expression, C2C12 cells were seeded at 1×10⁵ onto one 6-well plate and cultured for 24 h in DMEM containing 10% FBS, 1% penicillin/streptomycin, and 1% glutamine. Cells were then infected with AAVBMP2 at a MOI (Multiplicity of Infection: viral particles/cell) of 10⁶ and 0 (mock) for 24 h, washed with PBS, fixed with 3.7% formaldehyde in PBS for 10 min, permeabilized with 0.1% Triton X-100 in PBS for 10 min, then incubated with a mouse monoclonal antibody against human BMP2 (Sigma, St. Louis, Mo., USA) in PBS-3% bovine serum albumin (BSA) overnight at 4° C. Cells were then thoroughly washed with PBS and stained with fluorescein isothiocyanate (FITC) conjugated goat anti-mouse IgG at 1:100 dilutions (Zymed Laboratories Inc., San Francisco, Calif., USA) for 2 h in the dark, washed with PBS, and mounted for observation under fluorescence microscopy.

6.3. In Vitro Transduction of AAV-BMP2 in Mouse Myoblast C2C12 Cells

Mouse myoblast C2C12 cells were cultured at 37° C. under 5% CO₂ in DMEM medium containing 10% FBS, 1% penicillin/streptomycin and 1% glutamine. Cells were plated at 2×10⁵ cells/well on 6-well plates, allowed to reach 70% confluence. Then, cells were exposed to varied doses of AAV-BMP2 at MOI (particles/cell) of 2×10⁶, 1×10⁶, 5×10⁵ and 0 (mock). Control cells were exposed to AAV-EGFP at a MOI of 1×10⁶ (each group is composed of 3 wells). Twenty-four hours later, cells were rinsed with PBS and growth medium was replaced with fresh medium. Six days after infection, the phenotype changes of C2C12 cells were observed under conventional microscopy. To measure BMP2 secretion rate, the conditioned medium from either AAV-BMP2 (MOI of 10⁶ and 0) or AAV-EGFP (MOI of 10⁶) treated C2C12 cells was collected, and the secretion of BMP2 protein in the medium was determined by a commercially available human BMP2 ELISA Kit (R&D Systems, Inc., Minneapolis, Minn., USA). For osteoblastic differentiation analysis, cell layers were rinsed with PBS and lysed by a buffer containing 50 mM Tris-HCl and 0.5% NP-40 at pH 7.5. The cellular alkaline phosphatase (ALP) activity, an important osteoblastic differentiation marker, was determined by the pNPP hydrolosis method using the ALP Assay Kit (Upstate Biotechnology, Lake Placid, N.Y., USA).

6.4. In Vivo Gene Transfer for Bone Formation

Twelve male immuno-competent Sprague-Dawley (SD) rats aging between 5˜7 weeks were used in this study. All animal procedures were in accordance with Animal Ordinance, established by Department of Health, Hong Kong Regional Office. Animals were randomly assigned into three groups. Group I contains six animals treated with AAVBMP2 (10¹² viral particles, VP). Group II contains three animals treated with AAV-EGFP (10¹² VP), Group III contains three animals treated with empty AAV (10¹² VP). After adequately anesthetized with a mixture of ketamine (90 mg/kg, i.p.) and xylazine (10 mg/kg, i.p.), a 5 mm incision was made on the right hind limb that was prepared in a sterile fashion. Then, either AAV-BMP2, AAV-EGFP or empty AAV vector was injected directly into the musculature using a micro-syringe (Hamilton, Reno, Nev., USA). One interrupted 4-0 silk suture was used to close the incision. Animals were allowed ad libitum activity, food and water after the injection. Animals were radiographically examined at 2, 3, 4, 5, 6, and 8 weeks after operation. At 1, 3, 8 weeks post-injection, SD rats were killed by administration of a fatally high dose of anesthetics, and the new bone tissues at the injection site were performed histological analysis. Hind limbs were harvested so that the posterior musculature could then be dissected from the rest of hind limb, and the muscle samples were stored at −20° C. until ready for histological analysis. The harvested ossified tissues were fixed in 10% formalin neutral buffer solution at pH 7.4 for 2 days, and then decalcified with decalcifying solution composed of 10% HCl and 0.1% ethylenediamine tetraacetic acid for another 2 days. The specimens were then dehydrated through a series of graded ethanol, followed by infiltration and embedding into paraffin wax. The tissues were cut into 10 μm sections and stained with hematoxylin and eosin.

### 7. Results

7.1. Expression and Secretion of BMP2 in Mouse Myoblast C2C12 Cells

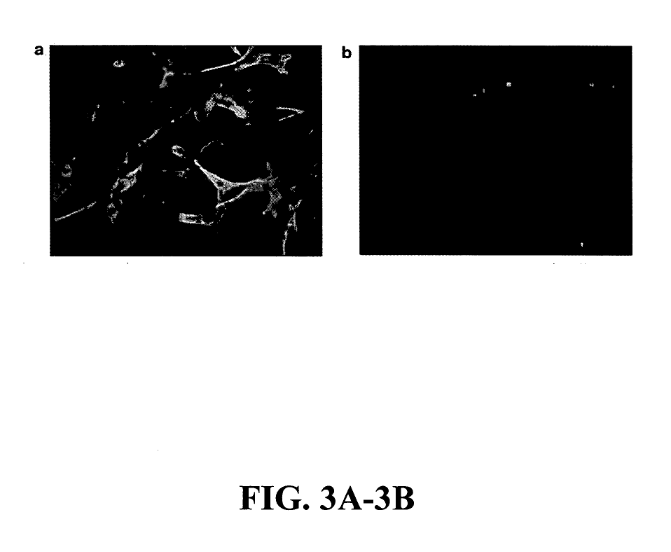

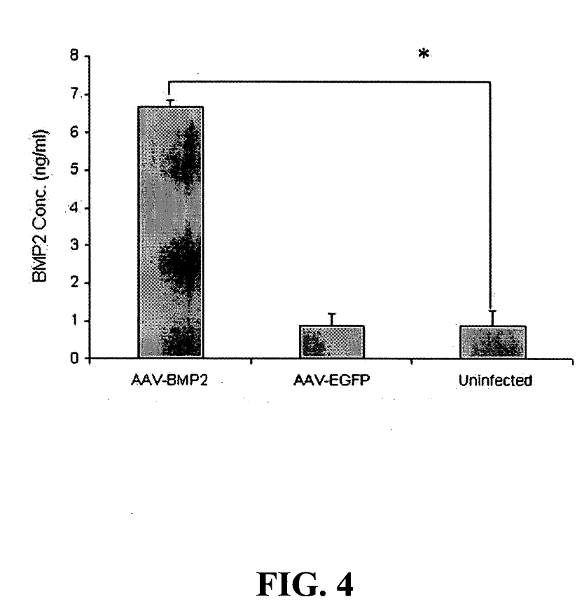

To demonstrate the gene transfer of BMP2 by the AAV vector, we transduced myoblast C2C12 cells with AAV-BMP2 at a MOI (Multiplicity of Infection: particles/cell) of 10⁶ and 0 (mock) as described in Section 6. Twenty-four hours after infection with AAV-BMP2, the BMP2 expression in the C2C12 cells were visualized by immunofluorescence staining using the monoclonal antibody against human BMP2. Under fluorescence microscopy, only those C2C12 cells infected with AAV-BMP2 showed positive BMP2 staining (FIG. 3A), which was mainly located in the cytoplasm. However, those uninfected C2C12 cells showed negative immunofluorescence staining (FIG. 3B). In addition, six days after infection of AAV-BMP2 or AAV-EGFP at a MOI of 10⁶, the conditioned medium was collected for measurement of secreted BMP2 protein using ELISA. As shown in FIG. 4, the AAV-BMP2 infected C2C12 cells showed a significantly higher level of BMP2 protein secretion, as compared to that obtained from the AAV-EGFP infected cells and uninfected cells.

7.2. Phenotype Changes of C2C12 Cells Transduced with AAV Vectors

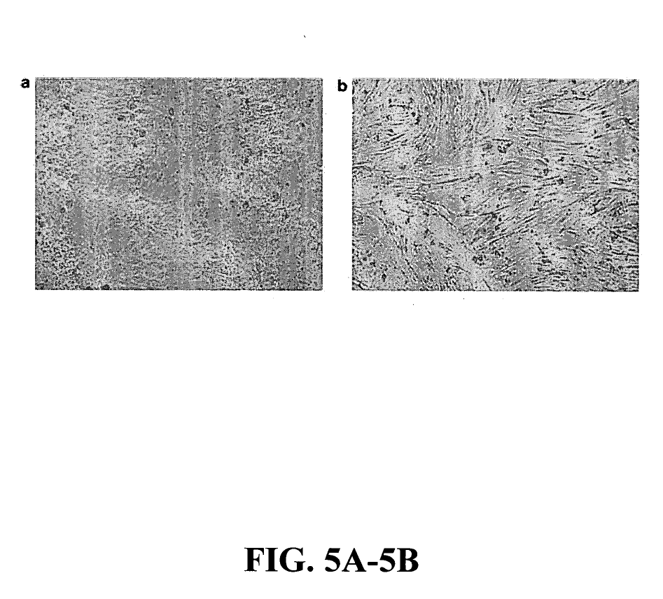

Six days after infection of AAV-BMP2 or AAV-EGFP at a MOI (particles/cells) of 10⁶ and 0 (mock), the phenotype of C2C12 cells revealed a significant change. Those uninfected C2C12 cells or those transduced with AAV-EGFP generated further myogenic differentiation, as confirmed by formation of numerous multinucleated myotubes on day 6 (FIG. 5B). However, myoblast C2C12 transduced with AAV-BMP2 inhibited their myotube formation, and almost all of these cells remained as unfused mononuclear round-like or polygonal cells, a typical osteoblast cell phenotype (FIG. 5A).

7.3. Osteogenic Induction Activity in AAV-BMP2 Infected C2C12 Cells

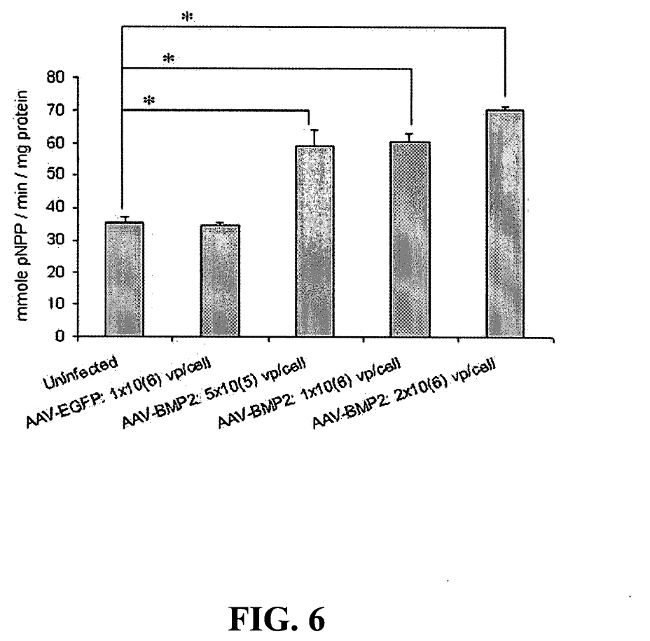

To demonstrate the osteogenic induction activity of the secreted BMP2, alkaline phosphatase (ALP) activity was measured, an important osteoblastic differentiation marker, in C2C12 cell layers on day 6 after infection (FIG. 6). Although there was intrinsic expression of ALP, C2C12 cells infected with AAV-BMP2 still showed a significant increase of ALP activity, as compared to those infected with AAV-EGFP or uninfected cells. Combining with phenotype changes, after treatment with AAV-BMP2, C2C12 cells underwent an obvious differentiation change from myoblast into osteoblast.

7.4. Radiographic and Histological Evidences for AAV-BMP2 Mediated In Vivo New Bone Formation in SD Rats

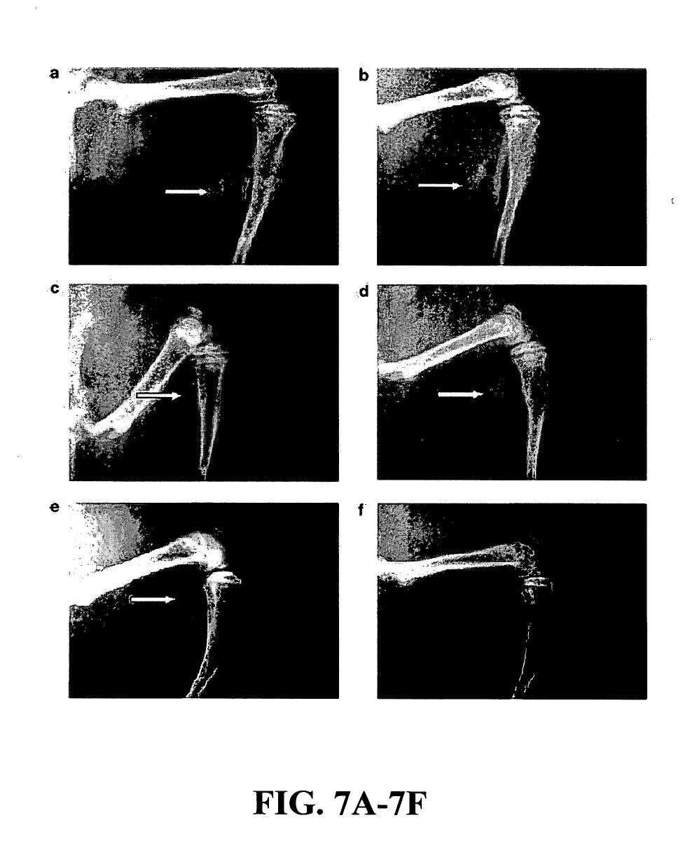

Immuno-competent SD rats were intramuscularly injected with AAV-BMP2 (10¹² viral particles, VP), AAV-EGFP (10¹² VP), and empty AAV (10¹² VP) into their hind limb to produce new bone. X-ray films were taken in those animals at 2, 3, 4, 5, 6, and 8 weeks post-injection. At 1, 3, 8 weeks after operation, rats were sacrificed and the newly formed bone tissues were examined by histological analysis. X-rays films showed that significantly visible bone tissue could be produced in all those rats receiving AAV-BMP2 as early as 3 weeks post-injection (FIGS. 7A and 7C). Although the intensity of new bone mass increased at 8 weeks post-injection, whose size did not reveal any significant difference from that of rats at 3 weeks post-injection (FIGS. 7B and 7D). In contrast, no radiographic evidence of bone formation was present in rats injected with either AAV-EGFP (FIG. 7F) or empty AAV (data not shown) at all time intervals.

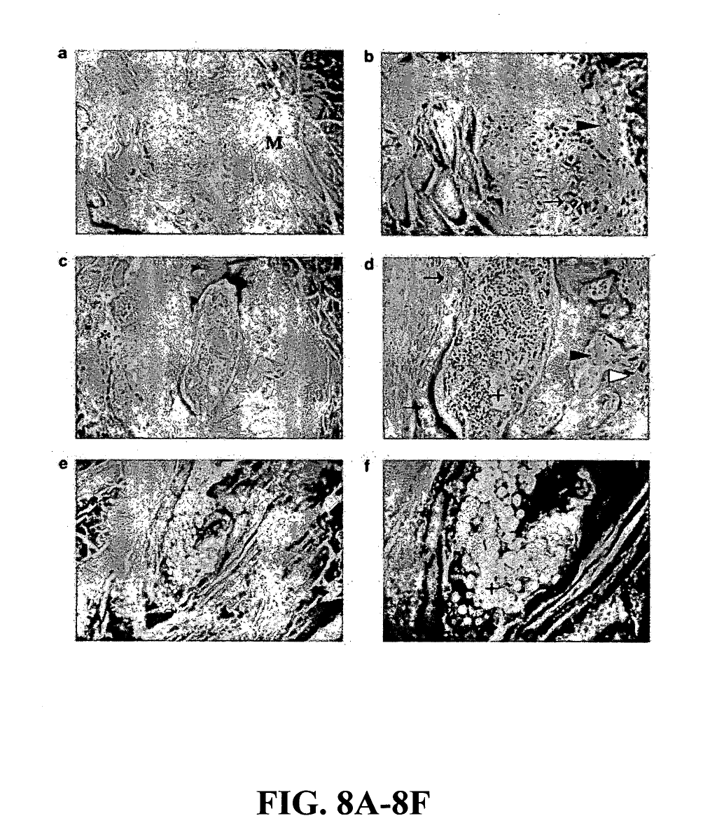

Standard hematoxylin-eosin staining confirmed the formation of new bone only in rats receiving AAV-BMP2. At one week post-injection, there was a significant accumulation of chondrocytes within the muscle tissue, which produced a loose extracellular matrix. The cartilaginous mass was surrounded by undifferentiated mesenchymal cells (FIGS. 8A and 8B). At three weeks post-injection, woven bone was formed which was characterized by a well-defined perimeter of cortical bone rim with trabeculae structure, an obvious marrow cavity containing bone marrow and adipocyte-like cells. Other differentiated structure including osteocytes, osteoblasts, and osteoclasts were also presented within bone forming area. In addition, muscle tissue surrounding the new bone showed some fatty degeneration (FIGS. 8C and 8D). At eight weeks post-injection, more mature bone was formed, with an enlarged medullary cavity. The bone matrix showed a high lamellar structure, and the predominant cell type is osteocyte (FIGS. 8E and 8F). No bone tissue structure was found in rats receiving either AAV-EGFP or PBS (data not shown). In addition, it is noticeable that there was no mononuclear or macrophage cell infiltration, which indicated that no detectable immunological responses were presented. Moreover, during the whole experiment procedure, all animals survived well until the scheduled date of killing with no apparent complications.

7.5. Radiographic Analysis of New Bone Formation in Animals Injected with Both AAVBMP2 and Ad-BMP2

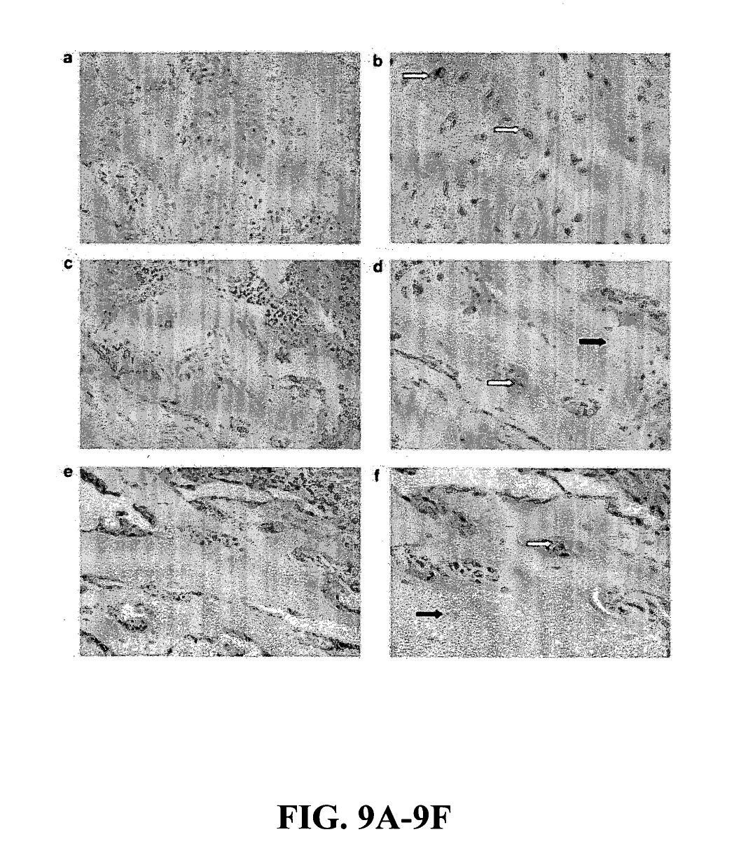

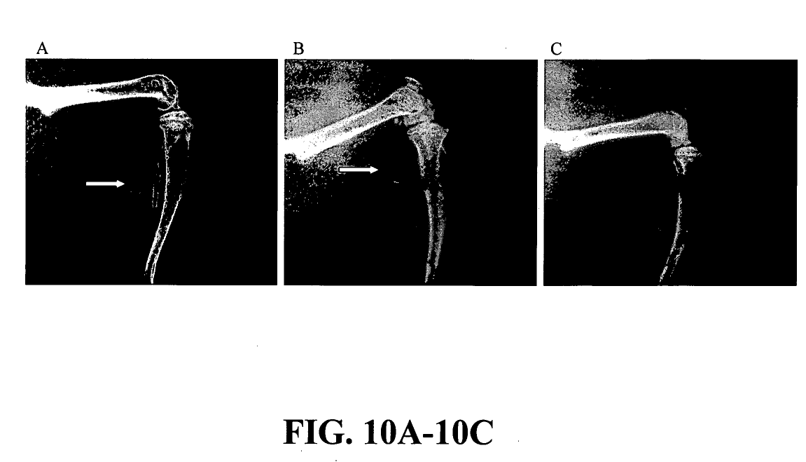

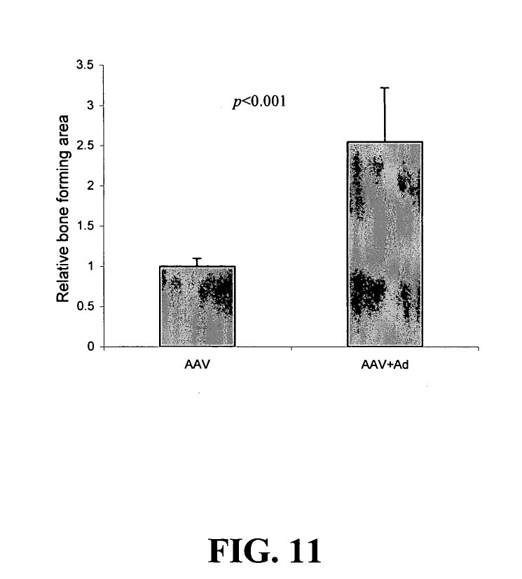

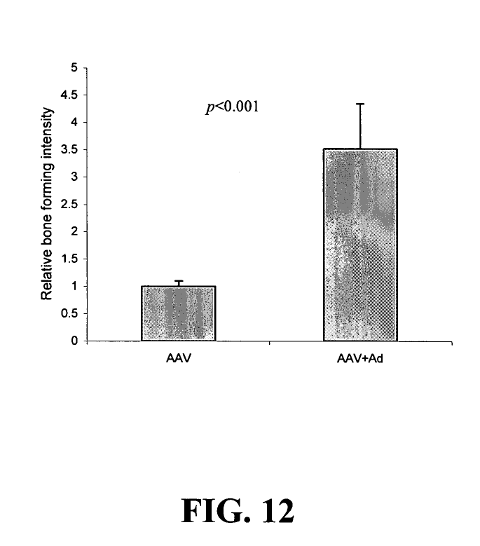

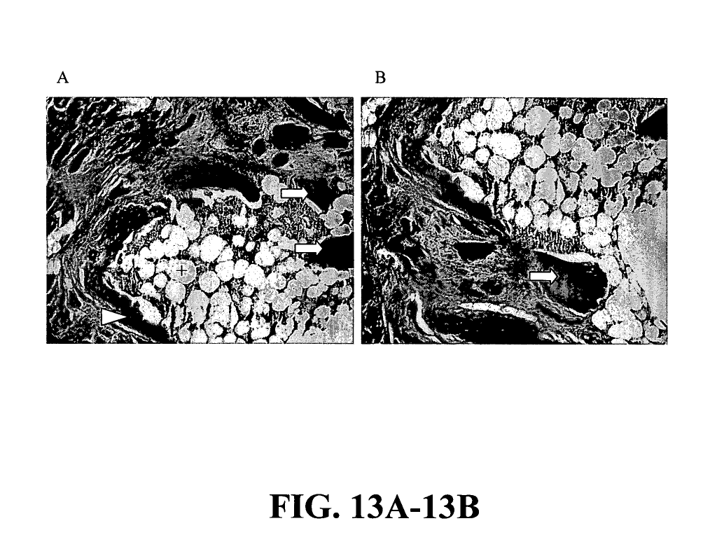

Examination of X-ray films detected that all those animals that had been co-injected with AAV-BMP2 (5×10¹¹ VP) and low level of Ad-BMP2 (5×10⁸ VP) had developed significant bone formation within the hind limb muscles (FIG. 10A), whereas those animals injected with AAV-BMP2 (5×10¹¹ VP) alone displayed only mild ossification (FIG. 10B). Radiographic morphometric analysis revealed a much significant increase of bone-forming area and bone-forming intensity in animals injected with both AAV-BMP2 and Ad-BMP2, comparing to those treated with AAV-BMP2 alone (FIGS. 10A-10C, 11, and 12). Moreover, histological analysis using hematoxylin and eosin staining in tissue sections demonstrated an increased bone forming region and an enlarged medullary cavity in co-injected animals. Also, it was noticeable that there was no significant infiltration of lymphocytes in tissue sections, which suggested that no severe immune responses had taken place (FIGS. 13A and 13B; also see FIGS. 9A-9F).

In contrast, there was neither radiographic nor histological evidence for bone formation in animals treated with AAV-EGFP (data not shown). Hence, AAV-BMP2-mediated osteogenic activity is enhanced by co-injection with low level Ad-BMP2 without pronounced immunogenicity.

### 8. Discussion

The present invention provides AAV-mediated BMP2 gene therapy for new bone formation both in vitro and in vivo in immunocompetent rats. AAV-BMP2 gene delivery is an efficient and safe procedure for the treatment of fracture non-union, segmental bone defects, spinal fusion, or other areas requiring bone augmentation.

An expanded role for gene therapy in the musculoskeletal system is important, particularly, in light of the recent advancement of our knowledge concerning the proteins that are responsible for the growth and regeneration of tissues, such as bone, cartilage, muscle, and ligament. It was first shown by Fang and colleagues in 1996 that stimulation of new bone formation by direct transfer of BMP4 plasmid vector could be achieved in rat femoral segmental defect model (Fang et al., 1996, Proc Natl Acad Sci 93:5753-5758). Several methods have been developed to increase the transfection rate of plasmid vectors, these include the use of gene gun (Klein et al., 1992, Biotechnology 24:384-386), a combination of plasmid DNA with liposomes (Goomer et al., 2001, Osteoarthritis Cartilage 9:249-256), or the use of gene-activated matrix (Fang et al., 1996, Proc Natl Acad Sci 93:5753-5758; Bonadio et al., 1999, Nat Med 5:753-759). However, the efficiency of gene delivery by plasmid vectors is still very poor. Furthermore, since DNA is not incorporated into the host nuclear DNA, the time of expression is merely transient. So far, the most efficient vectors for gene delivery are viruses. Retroviruses vectors have been used in human trials since 1991. Retrovirus mediated interleukin-1 (IL-1) receptor antagonist is currently the only human clinical trial being conducted in orthopedics-related gene therapy (Evans et al., 1996, Hum Gene Ther 7:1261-1280). It has been reported that transplantation of allogenic osteoprogenitor cells transduced with a retroviral vector encoding BMP2 can successfully induce bone formation (Engstrand et al., 2000, Hum Gene Ther 11:205-211). Retroviral BMP7 vectors can also efficiently transduce cultured periosteal cells, and promote bone healing by ex vivo gene transfer technique (Breitbart et al., 1999, Ann Plast Surg 42:488-495). However, retroviruses can only infect dividing cells, and these viruses also insert themselves randomly into the host DNA, activating a cell proto-oncogene or disrupting a tumor suppressor gene and, therefore, retroviral gene transfer usually is applicable only in an ex vivo procedure.

The currently most commonly used vehicle for BMP gene therapy is the adenovirus vector (Ad), because it can infect both dividing and non-dividing cells with excellent efficiency. First generation adenoviral vectors containing BMP2 or BMP9 gene have been shown to induce osteogenesis in the thigh musculature of athymic nude rodents (Alden et al., 1999, Hum Gene Ther 10:2245-2253; Varady et al., 2001, Hum Gene Ther 12:697-710). Histological examination of the injected region showed clear evidence of endochondral bone formation as early as 3 weeks after treatment. The feasibility of ex vivo technique employing Ad-BMP2 as a tool has also been reported, in which segmental bone defects healing was successfully achieved in rat model with human BMP2-producing bone marrow cells (Lieberman et al., 1999, J Bone Joint Surg 81A:905-917). Furthermore, Ad-BMP7, mixed with a collagen carrier, could also induce clearly defined bone tissue after implantation into mouse muscle (Franceschi et al., 2000, J Cell Chem 78:476-486). More recently, in vitro and in vivo osteogenic activity of Ad-BMP4 gene therapy has been monitored (Chen et al., 2002, Bioichem Biophys Res Commun 298:121-127). Application of adenovirus vector is greatly limited by the lack of persistent expression and by severe immune response in immunocompetent animals (Jiang et al., 2001, Mol Ther 3:892-900).

Mouse myoblast C2C12 cells transduced with recombinant AAV-BMP2 vector express and secrete high level of BMP2 protein in vitro, which was confirmed by immunofluorescence analysis and ELISA (see FIGS. 9A-9F). Untreated or AAV-EGFP infected myoblast C2C12 cells underwent a further myogenic differentiation, as confirmed by formation of multinucleated myotubes on day 6 (see FIG. 5B). However, in those treated with AAV-BMP2, the cells remained unfused mononuclear round-like or polygonal phenotype (see FIG. 5A), which is a typical characteristic phenotype of osteoblast lineage (Katagiri et al., 1994, J Cell Biol 127:1755-1766). Furthermore, AAV-BMP2-transduced cells demonstrated a significant increase of alkaline phosphatase (ALP) activity, an important osteoblastic marker. AAV-BMP2 transduction produces osteogenic BMP2 protein, which not only inhibits further myogenic differentiation of C2C12, but also converts their differentiation pathway of myoblast into that of osteoblast lineage.

More importantly, after direct injection of AAV-BMP2 into musculature of SD rats, radiographic data demonstrated that new bone formation was induced as early as 3 weeks. Examination of the histological specimens revealed a well-defined perimeter of cortical bone with trabeculae and an obvious medullary cavity within the skeletal muscle. Differentiated structures also include many osteocytes, osteoblasts and osteoclasts as well. Although the newly formed bone became more mature at 8 weeks post-injection indicated by highly differentiated lamellar cortical bone structure, little difference of bone size was noted between these two time intervals. In addition, consistent with the previous report that BMP2 protein induces endochondral bone formation in vivo (Wozney et al., 1998, Bone 22:591-603), there was a significant accumulation of chondrocytes within the muscle tissue at one week post-injection, which produced a loose extracellular cartilaginous matrix. Therefore, AAV-BMP2 activates an endochondral mechanism for in vivo bone formation.

Direct intramuscular injection of Ad-BMP2 or Ad-BMP4 induces significant new bone as early as three weeks post-injection in athymic nude rats (Sandhu et al., 1999, Bone 24:217-227). Direct transfer of AAV-BMP2 into immunocompetent SD rats successfully induced radiographic ossicification as early as three weeks post-injection. Histological examination further demonstrated a typical bone remodeling structure without obvious mononuclear cell infiltration. In addition, rats injected with AAV-BMP2 survived well through the whole experiment procedure without any complication.

Furthermore, direct injection of AAV-BMP2 vectors into skeletal muscle scaffold is a novel approach to bone augmentation. Skeletal muscle is a useful target for gene delivery approach because of its large mass, vascularity and accessibility. Since muscle fibers are non-dividing, effective gene delivery could potentially result in long-lived protein production. Posttranslational modification of proteins in muscle cells allows these proteins to be secreted with full potency and bioavailability (Blau et al., 1995, N Engl J Med 333:1554-1556). Although the basal lamina surrounding mature muscle fibers has pores of 40 nm in size and acts as a relative barrier to direct viral gene therapy with adenovirus, herpes simplex virus, and retroviruses vectors whose sizes are all larger than 40 nm (Yurchenco et al., 1990, Ann N Y Acad Sci 580:195-213), this hurdle can be overcome by using AAV vectors. Because AAV has very small viral particle size (20 nm), and can easily bypasses extracellular barriers (e.g. basal lamina), therefore facilitates efficient transduction in skeletal muscle system (Pruchnic et al., 2000, Hum Gene Ther 11:521-536). In addition, tropism of adenovirus vectors for muscle fibers has been observed to decline with muscle maturation, during which process myofibers down-regulate expression of cellular receptors for adenovirus (Nalbantoglu et al., 1999, Hum Gene ther 10: 1009-1019).

As a result, infection of immature or regenerating muscle by adenovirus is more efficient than infection of mature muscle. AAV, in contrast, seems to infect mature muscle as efficiently as immature muscle (Snyder et al., 1997, Hum Gene Ther 8:1891-1900). After directly injected into skeletal muscle, AAV-BMP2 vectors could efficiently transduce muscle fibers, which express and secrete osteogenic BMP2 protein. The extracellular BMP2 subsequently stimulates pluripotent mesenchymal cells to migrate to the injection site and proliferate between the muscle fibers. These cells then differentiate into small chondrocytes that produce a loose cartilaginous matrix, and the matrix calcifies to form woven bone, which finally remodels into normal lamellar bone (Sakou et al., 1998, Bone 22:591-603; Reddi et al., 1981, Coll Relat Res 1:209-226). As to the source of osteoprogenitors responsible for new bone formation within skeletal muscle, it has been shown that muscle-derived stem cell (MDSC) stimulated by BMP2 protein undergoes osteoblastic differentiation both in vitro and in vivo (Lee et al., 2000, J Cell Biol 150:1085-1099). Therefore, it is possible that these MDSCs may play an important role during AAV-BMP2 mediated intramuscular bone formation. In general, our studies have provided evidence that skeletal muscle can successfully function as an ideal bone substitute after injected with AAV-BMP2 vectors, which will be much more useful for treatment of many orthopedic disorders, in which bone augmentation is required.

One important issue is whether there is a threshold dosage of AAV-BMP2 for effective osteoinductive activity. It was indicated that rats injected with AAV-BMP2 at a dosage of 5×10¹¹ VP showed very mild ossicification. Injection of AAV-BMP2 at a dosage of 10¹² VP could induce significantly visible bone tissue. The bone volume can still be increased if higher dosages of AAV-BMP2 vectors are to be injected. In addition, being a secreted protein, BMP2 is expected to exert its osteoinductive activity only locally in the extracellular matrix. Although the new bone became more mature at 8 weeks post-injection, characterized by lamellar cortical bone matrix, the size of ossicification tissue showed no significant difference from that of animals at 3 weeks post-injection. This shows that the newly formed bone volume was relatively stable. Recently, it has been reported that the use of a tetracycline-inducible promoter allows regulation of AAV-based therapeutic interleukin-10 (IL-10) gene expression after intramuscular injection into mice model of rheumatoid arthritis (Apparailly et al., 2002, Hum Gene Ther 13:1179-1188). AAV vector may be modified by addition of BMP2 promoter which results in controlled BMP gene therapy. The present invention encompasses the introduction of a cell-type specific promoter in the recombinant AAV vector, which can target controlled transgene (e.g., BMP2) expression to specific cell types in musculoskeletal system.

Several in vitro studies have reported that the transduction efficiency of rAAV vectors can be significantly augmented by simultaneous treatment with genotoxic chemicals such as hydroxyurea, or with some physical agents such as UV (Yakobson et al., 1989, J. Virol. 63:1023-1030; Yakobson et al., 1987, J. Virol. 61:972-981). By using AAV-LacZ, Ferrari (Ferrari et al. 1996, J. Virol. 70:3227-3234) demonstrated a 100- and 1000-fold increase in transduction frequency when 293 cells were co-infected with adenovirus. Fisher (Fisher et al. 1996, J. Virol. 70:520-532) also reported that adenovirus could dramatically enhance rAAV transduction in vitro. Although AAV-BMP2 at a dosage of 5×10¹¹ VP was capable of initiating bone induction cascade, the newly formed bone tissue was only mild under radiographic and histological analysis. In contrast, after co-injection of same dosage AAV-BMP2 with low dosage Ad-BMP2 into the muscles of immunocompetent SD rats, more significant ossification tissue had successfully developed. Radiographic morphometric analysis indicated that either bone-forming area or bone mass intensity had increased greatly comparing to that obtained in animals treated with same dosage of AAV-BMP2 alone. Histological analysis further revealed an enlarged medullary cavity without severe lymphocytes infiltration in co-injected rats.

The mechanism whereby Ad-BMP2 can promote rAAV mediated gene delivery is unclear. A previous study using transfection experiment with adenovirus genomic DNA suggested that the enhancement of adenovirus for in vitro rAAV transduction was not facilitated by adenovirus-mediated viral uptake, and was not dependent on the rAAV gene cassette, but was instead dependent on adenovirus gene expression (Ferrari et al., 1996, J. Virol. 70:3227-3234). For example, it has been reported that the early-region E4 open reading frame 6 (E4 ORF6) is involved in an immediate early step of the AAV life cycle, namely, second-strand synthesis. Since AAV is a non-enveloped virus with a single-stranded linear DNA genome, and the synthesis of a second strand is a rate-limiting step for transduction of therapeutic genes by current rAAV vectors, E4 ORF6 provided by adenovirus could have significant effect on the use of rAAV vectors in gene therapy. Furthermore, it has also been documented that the rate-limiting step was substantially enhanced by expression of adenovirus genes E1 and E4, and that E4 ORF6 was sufficient and necessary to enhance rAAV transduction significantly (Fisher et al., 1996, J. Virol. 70:520-532). Since E1 and E3 region of adenovirus in Ad-BMP2 had already been deleted, E4 ORF6 in Ad-BMP2 genome plays a pivotal role in the dramatic enhancement of AAV-BMP2 mediated transduction for bone formation. So far, the precise mechanism by which adenovirus interact with rAAV to enhance its transduction in vivo has not been defined.

Only low-level of Ad-BMP2 vectors was applied in the combinational gene transfer. In vivo gene transfer with Ad-BMP2 at a dosage of 10¹⁰ VP or Ad-BMP4 at a dosage of 4×10¹⁰ VP could exert great potential for new bone induction. However, those results were only obtained in athymic immunodeficient animals, and were not available in immunocompetent animals due to immune responses (Chen et al., 2002, Biochem. Biophys. Res. Commun. 98:121-127). Although Ad-BMP2 at a dosage of 5×10⁸ VP had indeed enhanced AAV-mediated bone induction, the dosage of Ad-BMP2 may be optimized for maximum synergistic effect. Under transient immunosuppression by the use of cyclophosphamide, Ad-BMP2 could successfully induce bone formation in immunocompetent rats without any severe inflammatory responses (Okubo et al., 2000, Biochem. Biophys. Res. Commun. 267:382-387). By using transient immunosuppressive method, the synergistic effects of combinational AAV-BMP2 and Ad-BMP2 gene therapy can be further improved.

The present invention provides AAV-BMP2 and Ad-BMP2 for in vivo bone induction. Low titer Ad-BMP2 greatly enhances AAV-BMP2 mediated transduction without incurring undesired immune response. This “synergistic effect” can overcome the conventional cumbersome and labor-intensive AAV production method, and offer a better strategy for bone augmentation in clinical trials.

Those skilled in the art will recognize, or be able to ascertain many equivalents to the specific embodiments of the invention described herein using no more than routine experimentation. Such equivalents are intended to be encompassed by the following claims. All publications, patents and patent applications mentioned in this specification are herein incorporated by reference into the specification to the same extent as if each individual publication, patent or patent application was specifically and individually indicated to be incorporated herein by reference in their entireties. Citation or discussion of a reference herein shall not be construed as an admission that such is prior art to the present invention.

What is claimed is:

1. A nucleic acid molecule comprising an adeno-associated viral vector and a promoter which is operably linked to a sequence encoding bone morphogenetic protein.

2. The nucleic acid molecule of claim 1, wherein said promoter is a promoter of bone morphogenetic protein.

3. The nucleic acid molecule of claim 1, wherein said promoter is a CAG promoter comprising a beta-actin promoter and a cytomegalovirus enhancer.

4. A nucleic acid molecule comprising an adeno-associated viral vector and a promoter which is operably linked to: (a) a nucleotide sequence of SEQ ID NO:1; or (b) a nucleotide sequence that encodes the amino acid sequence of SEQ ID NO:2.

5. The nucleic acid molecule of claim 4, wherein said promoter is a promoter of bone morphogenetic protein.

6. The nucleic acid molecule of claim 4, wherein said promoter is a CAG promoter comprising a beta-actin promoter and a cytomegalovirus enhancer.

7. A vector comprising the nucleic acid molecule of any one of claims 1, 2, 3, 4, 5 or 6.

8. A host cell comprising the nucleic acid molecule of claim 7.

9. A pharmaceutical composition comprising the nucleic acid molecule of any one of claims 1, 2, 3, 4, 5 or 6; and a pharmaceutically acceptable carrier.

10. A method of treating a disease or disorder in a subject in need thereof, said method comprising administering to said subject a therapeutically effective amount of a nucleic acid molecule comprising an adeno-associated viral vector and a promoter which is operably linked to a sequence encoding bone morphogenetic protein.

11. The method of claim 10, wherein said promoter is a promoter of bone morphogenetic protein.

12. The method of claim 10, wherein said promoter is a CAG promoter comprising a beta-actin promoter and a cytomegalovirus enhancer.

13. A method of treating a disease or disorder in a subject in need thereof, said method comprising administering to said subject a therapeutically effective amount of a nucleic acid molecule comprising an adeno-associated viral vector and a promoter which is operably linked to: (a) a nucleotide sequence of SEQ ID NO:1; or (b) a nucleotide sequence that encodes the amino acid sequence of SEQ ID NO:2.

14. The method of claim 13, wherein said promoter is a promoter of bone morphogenetic protein.

15. The method of claim 13, wherein said promoter is a CAG promoter comprising a beta-actin promoter and a cytomegalovirus enhancer.

16. The method of claim 13 wherein the nucleic acid molecule is administered to a muscle of said subject.

17. A pharmaceutical composition comprising a first nucleic acid molecule comprising an adeno-associated viral vector and a first promoter which is operably linked to a nucleotide sequence encoding bone morphogenetic protein; a second nucleic acid molecule comprising an adenoviral vector and a second promoter which is operably linked to a nucleotide sequence encoding bone morphogenetic protein; and a pharmaceutically acceptable carrier.

18. The pharmaceutical composition of claim 17, wherein said first promoter and/or said second promoter is a promoter of bone morphogenetic protein.

19. The pharmaceutical composition of claim 17, wherein said first promoter and/or said second promoter is a CAG promoter comprising a beta-actin promoter and a cytomegalovirus enhancer.

20. A host cell comprising a first nucleic acid molecule comprising an adeno-associated viral vector and a first promoter which is operably linked to a nucleotide sequence encoding bone morphogenetic protein; and a second nucleic acid molecule comprising an adenoviral vector and a second promoter which is operably linked to a nucleotide sequence encoding bone morphogenetic protein.

21. The host cell of claim 20, wherein said first promoter and/or said second promoter is a promoter of bone morphogenetic protein.

22. The host cell of claim 20, wherein said first promoter and/or said second promoter is a CAG promoter comprising a beta-actin promoter and a cytomegalovirus enhancer.

23. A method of treating a disease or disorder in a subject in need thereof, said method comprising administering to said subject a therapeutically effective amount of a first nucleic acid molecule comprising an adeno-associated viral vector and a first promoter, and a second nucleic acid molecule comprising an adenoviral vector and a second promoter, wherein the first and second promoters are each operably linked to either: (a) a nucleotide sequence of SEQ ID NO:1; or (b) a nucleotide sequence that encodes the amino acid sequence of SEQ ID NO:2.

24. The method of claim 23, wherein said first promoter and/or said second promoter is a promoter of bone morphogenetic protein.

25. The method of claim 23, wherein said first promoter and/or said second promoter is a CAG promoter comprising a beta-actin promoter and a cytomegalovirus enhancer.

26. The method of claim 23 wherein said first and second nucleic acid molecules are administered to a muscle of said patient.

27. A pharmaceutical composition comprising a first nucleic acid molecule comprising an adeno-associated viral vector and a first promoter which is operably linked to a nucleotide sequence encoding a polypeptide; a second nucleic acid molecule comprising an adenoviral vector and a second promoter which is operably linked to a nucleotide sequence encoding the polypeptide; and a pharmaceutically acceptable carrier.

28. A host cell comprising a first nucleic acid molecule comprising an adeno-associated viral vector and a first promoter which is operably linked to a first nucleotide sequence encoding a polypeptide; and a second nucleic acid molecule comprising an adenoviral vector and a second promoter which is operably linked to a second nucleotide sequence encoding a polypeptide.

29. A method of treating a disease or disorder in a subject in need thereof, said method comprising administering to said subject a therapeutically effective amount of a first nucleic acid molecule comprising an adeno-associated viral vector and a first promoter which is operably linked to a first nucleotide sequence encoding a polypeptide; and a second nucleic acid molecule comprising an adenoviral vector and a second promoter which is operably linked to a second nucleotide sequence encoding a polypeptide.

30. The method of claim 29 wherein the amount of the first nucleic acid molecule is higher than the amount of the second nucleic acid molecule.


SEQUENCE LISTING (2 sequences)

SEQ ID NO: 1 - DNA, 1547 bp, Homo sapiens
ggggacttcttgaacttgcagggagaataacttgcgcaccccactttgcgccggtgcctttgccccagcggagcctgcttcgccatctccgagccccaccgcccctccactcctcggccttgcccgacactgagacgctgttcccagcgtgaaaagagagactgcgcggccggcacccgggagaaggaggaggcaaagaaaaggaacggacattcggtccttgcgccaggtcctttgaccagagtttttccatgtggacgctctttcaatggacgtgtccccgcgtgcttcttagacggactgcggtctcctaaaggtcgaccatggtggccgggacccgctgtcttctagcgttgctgcttccccaggtcctcctgggcggcgcggctggcctcgttccggagctgggccgcaggaagttcgcggcggcgtcgtcgggccgcccctcatcccagccctctgacgaggtcctgagcgagttcgagttgcggctgctcagcatgttcggcctgaaacagagacccacccccagcagggacgccgtggtgcccccctacatgctagacctgtatcgcaggcactcaggtcagccgggctcacccgccccagaccaccggttggagagggcagccagccgagccaacactgtgcgcagcttccaccatgaagaatctttggaagaactaccagaaacgagtgggaaaacaacccggagattcttctttaatttaagttctatccccacggaggagtttatcacctcagcagagcttcaggttttccgagaacagatgcaagatgctttaggaaacaatagcagtttccatcaccgaattaatatttatgaaatcataaaacctgcaacagccaactcgaaattccccgtgaccagacttttggacaccaggttggtgaatcagaatgcaagcaggtgggaaagttttgatgtcacccccgctgtgatgcggtggactgcacagggacacgccaaccatggattcgtggtggaagtggcccacttggaggagaaacaaggtgtctccaagagacatgttaggataagcaggtctttgcaccaagatgaacacagctggtcacagataaggccattgctagtaacttttggccatgatggaaaagggcatcctctccacaaaagagaaaaacgtcaagccaaacacaaacagcggaaacgccttaagtccagctgtaagagacaccctttgtacgtggacttcagtgacgtggggtggaatgactggattgtggctcccccggggtatcacgccttttactgccacggagaatgcccttttcctctggctgatcatctgaactccactaatcatgccattgttcagacgttggtcaactctgttaactctaagattcctaaggcatgctgtgtcccgacagaactcagtgctatctcgatgctgtaccttgacgagaatgaaaaggttgtattaaagaactatcaggacatggttgtggagggttgtgggtgtcgctagtacagcaaaattaaatacataaatatatatata

SEQ ID NO: 2 - protein, 396 aa, Homo sapiens
MVAGTRCLLALLLPQVLLGGAAGLVPELGRRKFAAASSGRPSSQPSDEVLSEFELRLLSMFGLKQRPTPSRDAVVPPYMLDLYRRHSGQPGSPAPDHRLERAASRANTVRSFHHEESLEELPETSGKTTRRFFFNLSSIPTEEFITSAELQVFREQMQDALGNNSSFHHRINIYEIIKPATANSKFPVTRLLDTRLVNQNASRWESFDVTPAVMRWTAQGHANHGFVVEVAHLEEKQGVSKRHVRISRSLHQDEHSWSQIRPLLVTFGHDGKGHPLHKREKRQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR